(19) United States
(12) Patent Application Publication
(10) Pub. No.: US 2026/0085104 A1
(43) Pub. Date: Mar. 26, 2026

(54) METHODS OF DETECTING IMMUNE CELLS

(71) Applicant: Biosceptre (Aust) Pty Ltd, North Ryde, New South Wales (AU)

(72) Inventor: Patrick Schlegel, North Ryde, New South Wales (AU)

(21) Appl. No.: 19/110,332

(22) PCT Filed: Sep. 14, 2023
(86) PCT No.: PCT/AU2023/050889

§ 371 Date: Mar. 10, 2025

(30) Foreign Application Priority Data
Sep. 14, 2022 (AU) 2022902657

(51) Int. Cl. C07K 14/705; G01N 33/532; G01N 33/569

(52) U.S. Cl. CPC C07K 14/705; G01N 33/532; G01N 33/56972; C07K 2319/30; G01N 2333/705

(57) ABSTRACT

The invention relates to fusion proteins and uses thereof for obtaining and isolating enriched populations of immune cells. Exemplified fusion proteins include those comprising a dysfunctional purinergic P2X7 receptor (P2X7R) epitope moiety and a Fc region of an antibody.

### FIELD OF THE INVENTION

The present invention relates to in vitro methods for detecting immune cells, and compositions and molecules for performing same.

### RELATED APPLICATION

This application claims priority from Australian provisional application AU 2022902657, the entire contents of which are hereby incorporated by reference.

### BACKGROUND OF THE INVENTION

Cell therapy to treat cancer and other disease states is a rapidly growing field. The development of genetically modified immune effector cells such as T cells expressing chimeric antigen receptors (CARs) has revolutionised adoptive cell therapies.

The potential of this approach has been demonstrated in clinical trials, wherein CAR T cells were infused into adult and paediatric patients with B-cell malignancies, neuroblastoma, and sarcoma. To date, over 500 clinical trials have emerged worldwide, designed at testing the efficacy of CAR T cells targeted to bind 64 different tumour associated antigens. Among these, three CD19-specific CAR T cell products have been approved for the treatment of acute lymphoblastic leukaemia (ALL), large B cell lymphoma and mantle cell lymphoma. To date, most of the success with CAR T therapies has been observed in the context of so-called “liquid” tumours, or where the CARs are directed to CD19, CD22 or the B cell maturation antigen (BCMA).

Human derived T lymphocytes engineered to express CARs, which are expanded in in vitro culture and then infused into patients, exert robust cytotoxicity after tumor antigen recognition and subsequent activation. Various factors in the manufacture and administration of these cells contribute the in vivo persistence and durable antitumor effects of the CAR T cells.

Another important consideration during cellular immunotherapy protocols is the need to assess whether the CAR T cells continue to proliferate in vivo, following administration. This includes determining whether the cells continue to be present in the circulation of the patient at various time points following initial infusion of the cells.

There is therefore a need for methods and reagents for determining the presence of cellular immunotherapeutics, such as CAR T cells, in patient sample.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention finds particular application in the detection of genetically modified immune cells that are engineered to bind to dysfunctional P2X₇ receptor on a cancer cell. Accordingly, there is provided a fusion protein comprising:
(i) a dysfunctional P2X₇ receptor epitope moiety; and
(ii) an Fc region of an antibody, preferably wherein the Fc region has a reduced affinity for an Fc receptor compared to wild-type or naturally occurring Fc regions.

The present invention provides a fusion protein comprising:
(i) a peptide; and
(ii) an Fc region of an antibody, preferably wherein the Fc region has a reduced affinity for an Fc receptor compared to wild-type or naturally occurring Fc regions;
wherein the peptide comprises or consists of the amino acid sequence of SEQ ID NO: 7 (preferably the amino acid sequence of SEQ ID NO: 14). Optionally, the peptide comprises or consists of the amino acid sequence of any of SEQ ID NOs: 7 to 69 or 122 or sequences at least 80%, at least 81%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, provided that the sequences comprise at least the sequence set forth in SEQ ID NO: 14 or 7 or 9.

The Fc region of an antibody may be an Fc region of an IgG, IgA, IgD, IgE, or IgM. Preferably the Fc region is from an IgG antibody, such as an IgG1, an IgG2, an IgG2b, an IgG3 or an IgG4 antibody.

Preferably, the Fc region of the fusion protein comprises two heavy chain fragments, more preferably the CH2 and CH3 domains of said heavy chain.

The Fc region preferably comprises one or more amino acid substitutions for reducing affinity for the Fc receptor (FcR, including any of FcγRI, FcγRII and FcγRIII) and thereby reducing the ability of the fusion protein to elicit antibody-dependent cell-mediated toxicity (ADCC). Such substitutions are well known in the art and include, but are not limited to the “DANA” and “LALA” amino acid substitutions and variations thereof, as further defined herein.

In further embodiments, the Fc region may also comprise substitutions which abrograte recruitment of complement C1q. Such mutations are also well known in the art and are further described herein.

In preferred embodiments, the fusion protein has an affinity for FcR that is less than about 250 nM, preferably less than 500 nM, less than 1000 nM, most preferably less than 2000 nM.

In a preferred embodiment, the Fc region of the fusion protein comprises one or more amino acid substitutions compared to naturally occurring Fc sequences, which prevent or reduce the ability of the Fc region to homodimerise. Preferably, the amino acid substitutions comprise one or more substitutions of the cysteine residues so as to prevent the formation of disulphide bonds between Fc molecules. The cysteine residues of the Fc region may be substituted to any other amino acid residue, optionally to glycine, serine, alanine, lysine and glutamic acid, preferably glycine or serine.

The cysteine residues for substitution are preferably one or more of the cysteine residues located in the region of the Fc region which corresponds to the hinge region of an immunoglobulin. Examples of the IgG1 hinge regions, and variations thereof including cysteine to serine substitutions are provided herein in Table 2. The hinge region of an immunoglobulin (eg of IgG1) comprises three cysteine residues (which are number C220, C226 and C229 according to EU numbering. Accordingly, in any embodiment, at least one, at least two, or all three of the cysteine residues in the immunoglobulin hinge region are substituted. Preferably, at least two or all three of the cysteine residues are substituted. More preferably, all cysteine residues in the Fc region, such as the hinge region, are substituted. In particularly preferred embodiments, at least one of C226 and C229 are substituted, preferably wherein both C226 and C229 are substituted.

Accordingly, in preferred embodiments, the fusion protein comprises a hinge region for linking the peptide (eg a dysfunctional P2X₇ receptor epitope moiety) and Fc region of an antibody, wherein the hinge region comprises an amino acid sequence that corresponds to any of the sequences set forth in SEQ ID NOs: 76 to 113, or 136 to 137 or 141 or 142.

The present invention also provides a heterodimeric asymmetric molecule comprising a fusion protein as described herein (eg comprising a peptide of SEQ ID NO: 7 or 14, or variations thereof as exemplified in any of SEQ ID NOs: 2 to 69) and an Fc region of an antibody, and further comprising an Fc region of an antibody that does not comprise the peptide. Such asymmetric heterodimeric molecules may be obtained using the knob-in-hole technology, as further described herein, for facilitating dimerisation of non-identical Fc regions.

Preferably, the fusion protein or the heterodimeric asymmetric molecule, consists or consists essentially of the peptide and an Fc region of an antibody, such that the fusion protein or heterodimeric asymmetric molecule does not comprise an antigen binding domain of an antibody (ie such that the fusion protein does not comprise a VH, VL, Fab, Fv, or an scFv derived from an antibody).

In any embodiment, the peptide (eg dysfunctional P2X₇ receptor epitope moiety) may comprise any amino acid sequence which is derived from the dysfunctional P2X₇ receptor although preferably, comprises the sequence of an epitope which is found on dysfunctional P2X₇ receptor but not on functional P2X₇ receptor.

In a preferred embodiment, the amino acid sequence of the dysfunctional P2X₇ receptor epitope moiety comprises or consists at least of the amino acid sequence as set forth in SEQ ID NO: 14. In an especially preferred embodiment, the moiety comprises at least the sequence as set forth in SEQ ID NO: 7 or 9.

In any embodiment, the dysfunctional P2X₇ receptor epitope moiety comprises an amino acid sequence as set forth in any of SEQ ID NOs: 7 to 69, or 122, or sequences at least 80%, at least 81%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, provided that the sequences comprise at least the sequence set forth in SEQ ID NO: 14 or 7 or 9.

In any embodiment, the fusion protein comprises the amino acid sequence as set forth in any of SEQ ID NOs: 145 to 158, 160 or 161, or a sequence at least 80%, 85%, 86%, 87%, 88%, 89%, 90%, 91%, 92%, 93%, 94%, 95%, 96%, 97%, 98%, or 99% identical thereto.

In further embodiments, the fusion protein of the invention may comprise one or more modifications for enabling the detection of the fusion protein, including when the protein is bound to immune cells expressing an exogenous cell surface receptor comprising an antigen binding domain and an intracellular signalling domain (such as a chimeric antigen receptor (CAR) or modified TCR). Preferably, the antigen binding domain of the receptor is for binding to a peptide as described herein and/or for binding to a dysfunctional P2X₇ receptor.

It will be appreciated that typically the antigen binding domain of the receptor will be one which is capable of binding or recognising the same peptide (eg dysfunctional P2X₇ receptor epitope moiety) that is comprised in the fusion protein. Moreover, it is within the purview of the skilled person, having knowledge of the specific epitope and binding target of a given anti-dysfunctional P2X₇ receptor CAR, to be able to design a suitable fusion protein in accordance with the invention, for use in binding to the CAR.

The one or more modifications to the fusion protein may be selected from: a fluorescent moiety, a metallic particle (for use in Cytometric time of flight, CyTOF methods), a magnetic particle, a chromophore moiety, a phosphorescent moiety, a luminescent moiety, a light absorbing moiety, a radioactive moiety, and chemically detectable moieties like haptens, e.g. biotin, avidin, streptavidin and derivatives thereof.

In the case of biotin or magnetic moieties, the moieties may be conjugated to the fusion protein using any method known to the skilled person. In one example, the moieties are conjugated to the fusion protein via one or more lysine residues of the protein and/or at the amino-termini of the protein.

In a second aspect, the present invention also provides a use of a fusion protein of the first aspect, or a polypeptide as further described herein, for detecting one or more genetically modified immune cells which express a receptor that comprises an antigen binding domain for binding to dysfunctional P2X₇ receptor and/or a peptide as described herein Preferably, the receptor expressed by the immune cells is a chimeric antigen receptor (CAR), or optionally, a modified T cell receptor (TCR). Preferably the detecting is an in vitro method of detection, to enable determination of the presence of the immune cells in a complex mixture, such as a biological sample obtained from a patient who previously received treatment with the immune cells.

In a preferred embodiment of the second aspect, there is provided an in vitro method for detecting an immune cell expressing a receptor that comprises an antigen binding domain for binding to dysfunctional P2X₇ receptor, the method comprising:
(i) providing a biological sample from a patient who has received a treatment with immune cells, preferably immune effector cells, wherein the cells express a receptor comprising an antigen binding domain for binding to dysfunctional P2X₇ receptor;
(ii) contacting the sample with a polypeptide, wherein the polypeptide comprises an epitope of dysfunctional P2X₇ receptor which is recognised by the antigen binding domain of the receptor, and wherein the polypeptide comprises a detection moiety for enabling detection of the polypeptide;
   to thereby allow formation of a complex of the polypeptide bound to the cells;
(iii) detecting the complex, thereby detecting an immune cell expressing a receptor having an antigen binding domain for binding to dysfunctional P2X₇ receptor. Optionally the method comprises the step of first isolating the complex prior to the step of detecting.

Preferably, the cells are immune cells which express a chimeric antigen receptor (CAR) for binding to dysfunctional P2X₇ receptor. Accordingly, there is also provided an in vitro method for detecting immune cells expressing a chimeric antigen receptor (CAR) for binding to dysfunctional P2X₇ receptor, the method comprising:
(i) providing a biological sample from a patient who has received a treatment with immune cells, preferably immune effector cells, comprising a chimeric antigen receptor (CAR) for binding to dysfunctional P2X₇ receptor;
(ii) contacting the sample with a polypeptide, wherein the polypeptide comprises an epitope of dysfunctional P2X₇ receptor which is recognised by the CAR, and wherein the polypeptide comprises a detection moiety for enabling detection of the polypeptide;
   to thereby allow formation a complex of the polypeptide bound to the cells;
(iii) detecting the complex,
thereby detecting immune cells expressing a chimeric antigen receptor (CAR) for binding to dysfunctional P2X₇ receptor. Optionally the method comprises the step of first isolating the complex prior to the step of detecting.

there is provided an in vitro method for detecting an immune cell expressing an exogenous cell surface receptor comprising an antigen binding domain for binding to a peptide comprising or consisting of the amino acid sequence of SEQ ID NO: 7 or 14 (or optionally the amino acid sequence of any of SEQ ID NOs: 2 to 69 and 122), the method comprising:
(i) providing a biological sample from a patient who has received a treatment with immune cells, preferably immune effector cells, wherein the cells express an exogenous cell surface receptor comprising an antigen binding domain for binding to a peptide comprising or consisting of the amino acid sequence of SEQ ID NO: 7 or 14 (or optionally the amino acid sequence of any of SEQ ID NOs: 2 to 69 and 122);
(ii) contacting the sample with a polypeptide, wherein the polypeptide comprises the peptide that is recognised by the antigen binding domain of the receptor, and wherein the polypeptide comprises a detection moiety for enabling detection of the polypeptide;
   to thereby allow formation of a complex of the polypeptide bound to the cells;
(iii) detecting the complex, thereby detecting an immune cell expressing an exogenous cell surface receptor having an antigen binding domain for binding to comprising or consisting of the amino acid sequence of SEQ ID NO: 7 or 14 (or optionally the amino acid sequence of any of SEQ ID NOs: 2 to 69 and 122). Optionally the method comprises the step of first isolating the complex prior to the step of detecting.

Preferably, the cells are immune cells which express a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122). Accordingly, there is also provided an in vitro method for detecting immune cells expressing a chimeric antigen receptor (CAR) for binding to a peptide comprising or consisting of the amino acid sequence of SEQ ID NO: 7 or 14 (or optionally the amino acid sequence of any of SEQ ID NOs: 2 to 69 and 122), the method comprising:
(i) providing a biological sample from a patient who has received a treatment with immune cells, preferably immune effector cells, comprising a chimeric antigen receptor (CAR) for binding to the peptide;
(ii) contacting the sample with a polypeptide, wherein the polypeptide comprises the peptide which is recognised by the CAR, and wherein the polypeptide comprises a detection moiety for enabling detection of the polypeptide;
   to thereby allow formation a complex of the polypeptide bound to the cells;
(iii) detecting the complex,
thereby detecting immune cells expressing the chimeric antigen receptor (CAR). Optionally the method comprises the step of first isolating the complex prior to the step of detecting

In any embodiment of the second aspect of the invention, the polypeptide comprises the amino acid sequence of a fusion protein of any embodiment of the first aspect of the invention.

In any embodiment of the second aspect of the invention, the polypeptide comprises a first portion comprising a peptide (eg an epitope of dysfunctional P2X₇ receptor) joined to a further amino acid sequence for facilitating the solubility and stability of the first portion. The further amino acid sequence joined to the dysfunctional P2X₇ receptor epitope may comprise any suitable linker or hinge region, such as those exemplified in Tables 1 and 3. Such linker or hinge regions may comprise amino acid sequences comprised of glycine and serine repeats (so-called “GS” linker sequences, and variations thereof as further defined herein). The hinge region may also comprise sequences derived from the hinge region of an immunoglobulin, such as those defined in Table 3.

In further embodiments, the polypeptide may be in the form of a fusion protein comprising an epitope of dysfunctional P2X₇ receptor joined to a further amino acid sequence. The further sequence may comprise serum albumin, transferrin, a carboxy-terminal peptide of chorionic gonadotropin (CG) β chain, a non-exact repeat peptide sequence, a polypeptide sequence composed of proline-alanine-serine polymer, an elastin-like peptide (ELP) repeat sequence), a homopolymer of glycine residues or a gelatin-like protein.

Further still, the polypeptide may be in the form of a conjugate comprising a carbohydrate (such as polyethylene glycol (PEG)), a lipid, a liposome, a peptide, or an aptamer conjugated to the amino acid sequence comprising the epitope of dysfunctional P2X₇ receptor. In the case of a PEG conjugate, conjugation can be via activated carboxylic acids of the amino acid sequence comprising the epitope of dysfunctional P2X₇ receptor.

In accordance with the second aspect of the invention, the moiety for enabling detection of the polypeptide may be any suitable detectable moiety such as a fluorescent moiety, a magnetic particle, a chromophore moiety, a phosphorescent moiety, a luminescent moiety, a light absorbing moiety, a radioactive moiety, and chemically detectable moieties like haptens, e.g. biotin, avidin, streptavidin and derivatives thereof.

Optionally, where the polypeptide is labelled with a biotin moiety, the method may further comprise the step of contacting the cells (after step ii), with an anti-biotin antigen binding protein, preferably wherein the anti-biotin antigen binding protein comprises one or more moieties for enabling detection of the complex. Optionally the one or more moieties for enabling detection of the complex comprises a fluorophore (ie, such that the anti-biotin antibody is fluorescently labelled.)

Optionally, wherein the polypeptide comprises a magnetic label, the step of detecting may comprise i) applying a magnetic field to the population of cells; ii) removing or discarding the cells that are not attracted to the magnetic field, iii) removal of the magnetic field to thereby provide a population of immune cells expressing a chimeric antigen receptor (CAR) for binding to dysfunctional P2X₇ receptor.

In particularly preferred embodiments of the second aspect of the invention, the exogenous cell surface receptor that comprises an antigen binding domain (eg the chimeric antigen receptor. CAR), comprises an antigen binding domain that comprises the CDR amino acid sequences of PEP2-2-1 described in PCT/AU2010/001070 (WO2011020155, or in any one of the corresponding U.S. Pat. Nos. 9,127,059, 9,688,771, or U.S. Pat. No. 10,053,508). More preferably, the antigen binding domain of the receptor (eg CAR) comprises or consists of the amino acid sequence of the PEP2-2-1 antigen binding protein as described in PCT/AU2010/001070 (WO2011020155 or in any one of the corresponding U.S. Pat. Nos. 9,127,059, 9,688,771, or 10,053,508), incorporated herein by reference.

In any embodiment, the biological sample from a patient may be a sample of whole peripheral blood, or a derivative thereof, such a peripheral mononuclear monocyte (buffy coat) preparation.

In further embodiments, there is provided a kit for use in a method described herein, the kit comprising:
a fusion protein or polypeptide capable of being bound by a receptor (eg a CAR) for binding to dysfunctional P2X₇ receptor;
optionally, one or more reagents for enabling detection of the fusion protein or polypeptide and complexes thereof.

Optionally, the kit comprises written instructions for use in a method of the second aspect of the invention.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### Sequence Information

TABLE 1
exemplary sequences of dysfunctional P2X7 receptor and receptor
epitope moieties

| Peptide/protein | | SEQ ID |
|---|---|---|
| name | Sequence | NO: |
| Full length P2X₇ | MPACCSCSDVFQYETNKVTRIQSMNYGTIKWFFHVIIFSYVCFAL | 1 |
| receptor | VSDKLYQRKEPVISSVHTKVKGIAEVKEEIVENGVKKLVHSVFDT | |
| E200 and E300 | ADYTFPLQGNSFFVMTNFLKTEGQEQRLCPEYPTRRTLCSSDRG | |
| shown in bold | CKKGWMDPQSKGIQTGRCVVYEGNQKTCEVSAWCPIEAVEEAPR | |
| and underline | PALLNSAENFTVLIKNNIDFPGHNYTTRNILPGLNITCTFHKTQ | |
| Pro 210 shown in | NPQCPIFRLGDIFRETGDNFSDVAIQGGIMGIEIYWDCNLDRWF | |
| italics | HHCRPKYSFRRLDDKTTNVSLYPGYNFRYAKYYKENNVEKRTLI | |
| | KVFGIRFDILVFGTGGKFDIIQLVVYIGSTLSYFGLAAVFIDFL | |
| | IDTYSSNCCRSHIYPWCKCCQPCVVNEYYYRKKCESIVEPKPTL | |
| | KYVSFVDESHIRMVNQQLLGRSLQDVKGQEVPRPAMDFTDLSRL | |
| | PLALHDTPPIPGQPEEIQLLRKEATPRSRDSPVWCQCGSCLPSQ | |
| | LPESHRCLEELCCRKKPGACITTSELFRKLVLSRHVLQFLLLYQ | |
| | EPLLALDVDSTNSRLRHCAYRCYATWRFGSQDMADFAILPSCCR | |
| | WRIRKEFPKSEGQYSGFKSPY | |
| Exemplary E200 | GHNYTTRNILPGLNITC | 2 |
| epitope | | |
| Variant E200 | GHNYTTRNILPGLNIT | 3 |
| epitope (E200′) | | |
| E300 | KYYKENNVEKRTLIK | 4 |
| Variant E300 | KYYKENNVEKRTLIKVF | 5 |
| epitope (E300′) | | |
| Composite | GHNYTTRNILPGAGAKYYKENNVEK | 6 |
| E200/E300 | | |
| epitope | | |
| E200 epitope | GHNYTTRNILPGLNITS | 7 |
| Cys to Ser | | |
| modification | | |
| (“Core” E200 | | |
| sequence) | | |
| Extended E200 | GHNYTTRNILPGLNITSTFHK | 8 |
| Cys to Ser | | |
| modification | | |
| Extended E200′ | GHNYTTRNILPGLNITSTFHKT | 9 |
| Cys to Ser | | |
| modification | | |
| Extended E200″ | GHNYTTRNILPGLNITSTFHKTC | 10 |
| Cys to Ser | | |
| modification | | |
| Extended Pep17 | GHNYTTRNILPGLNITSTFHKTSGSGK | 11 |
| Pep17 | GHNYTTRNILPGLNITSTFHKTS | 12 |
| Extended E200′ | DFPGHNYTTRNILPGLNITSTFHKT | 122 |
| Cys to Ser | | |
| modification + N | | |
| term ext | | |
| Pep16 | DFPGHNYTTRNILPGC | 13 |
| Minimum E200 | NYTTRNILPGL | 14 |
| sequence | | |
| E200_G4S | GHNYTTRNILPGLNITSGGGGS | 15 |
| E200_2xG4S | GHNYTTRNILPGLNITSGGGGSGGGGS | 16 |
| E200_3xG4S | GHNYTTRNILPGLNITSGGGGSGGGGSGGGGS | 17 |
| E200_extended | GHNYTTRNILPGLNITSTFHKTGS | 18 |
| peptide 17v3 (24 | | |
| aa) | | |
| E200_extended | GHNYTTRNILPGLNITSTFHGS | 19 |
| peptide 17v4 (22 | | |
| aa) | | |
| E200_extended | GHNYTTRNILPGLNITSGS | 20 |
| peptide 17v5 (19 | | |
| aa) | | |
| E200_extended | DFPGHNYTTRNILPGLNITSGS | 21 |
| peptide 17v6 (22 | | |
| aa) | | |
| E200_extended | DFPGHNYTTRNILPGLNITSGGGGS | 22 |
| peptide 17v7 (25 | | |
| aa) | | |
| E200_extended | DFPGHNYTTRNILPGLNITSGGGGSGGGGS | 23 |
| peptide 17v8 (30 | | |
| aa) | | |
| E200_extended | DFPGHNYTTRNILPGLNITSGGGGSGGGGSGGGGS | 24 |
| peptide 17v9 (35 | | |
| aa) | | |
| E200_extended | DFPGHNYTTRNILPGLNITSTFHKTSGSGK | 25 |
| peptide 17v10 | | |
| (30 aa) | | |
| E200_extended | DFPGHNYTTRNILPGLNITSTFHKTSGSGKGS | 26 |
| peptide 17v11 | | |
| (32 aa) | | |
| E200_extended | DFPGHNYTTRNILPGLNITSTFHKTSGSGKGGGGS | 27 |
| peptide 17v12 | | |
| (35 aa) | | |
| E200_extended | DFPGHNYTTRNILPGLNITSTFHGGGGS | 28 |
| peptide 17v13 | | |
| (25 aa) | | |
| E200_extended | GHNYTTRNILPGLNITSTFHGGGGS | 29 |
| peptide 17v14 | | |
| (22 aa) | | |
| E200_extended | DFPGHNYTTRNILPGLNITSTFHKTGGGGS | 30 |
| peptide 17v15 | | |
| (30 aa) | | |
| E200_extended | GHNYTTRNILPGLNITSTFHKTGGGGS | 31 |
| peptide 17v16 | | |
| (27 aa) | | |
| E200 + IgG hinge | GHNYTTRNILPGLNITSEPKSSDKTHT | 32 |
| (E200 | | |
| underlined) | | |
| E200 + GS | GHNYTTRNILPGLNITSGSEPKSSDKTHT | 33 |
| linker_IgG hinge | | |
| E200 + G4S | GHNYTTRNILPGLNITSGGGGSEPKSSDKTHT | 34 |
| linker + IgG hinge | | |
| Extended E200 + | GHNYTTRNILPGLNITSTFHKTSGSGKEPKSSDKTHT | 35 |
| IgG hinge | | |
| Extended E200 + | GHNYTTRNILPGLNITSTFHKTSGSGKGSEPKSSDKTHT | 36 |
| GS linker + _IgG | | |
| hinge | | |
| Extended E200 + | GHNYTTRNILPGLNITSTFHKTSGSGKGGGGSEPKSSDKTHT | 37 |
| G4S linker + IgG | | |
| hinge | | |
| E200 + IgG | GHNYTTRNILPGLNITSEPKSSDKTHTGS | 38 |
| hinge + GS linker | | |
| E200 + GS | GHNYTTRNILPGLNITSGSEPKSSDKTHTGS | 39 |
| linker + IgG | | |
| hinge + GS linker | | |
| E200 + G4Slinker | GHNYTTRNILPGLNITSGGGGSEPKSSDKTHTGS | 40 |
| + IgG hinge + GS | | |
| linker | | |
| Extended | GHNYTTRNILPGLNITSTFHKTSGSGKEPKSSDKTHTGS | 41 |
| E200 + _IgG | | |
| hinge + GSlinker | | |
| Extended | GHNYTTRNILPGLNITSTFHKTSGSGKGSEPKSSDKTHTGS | 42 |
| E200 + GS | | |
| linker + _IgG | | |
| hinge + GS linker | | |
| Extended | GHNYTTRNILPGLNITSTFHKTSGSGKGGGGSEPKSSDKTHTGS | 43 |
| E200 + G4S | | |
| linker + _IgG | | |
| hinge + GS linker | | |
| E200 + IgG | GHNYTTRNILPGLNITSEPKSSDKTHTGGGGS | 44 |
| hinge + G4S linker | | |
| E200 + GS | GHNYTTRNILPGLNITSGSEPKSSDKTHTGGGGS | 45 |
| linker + IgG | | |
| hinge + G4S linker | | |
| E200 + G4S | GHNYTTRNILPGLNITSGGGGSEPKSSDKTHTGGGGS | 46 |
| linker + IgG | | |
| hinge + G4S linker | | |
| Extended | GHNYTTRNILPGLNITSTFHKTSGSGKEPKSSDKTHTGGGGS | 47 |
| E200 + IgG | | |
| hinge + G4S linker | | |
| Extended | GHNYTTRNILPGLNITSTFHKTSGSGKGSEPKSSDKTHTGGGGS | 48 |
| E200 + GS | | |
| linker + IgG | | |
| hinge + G4S linker | | |
| Extended | GHNYTTRNILPGLNITSTFHKTSGSGKGGGGSEPKSSDKTHTGG | 49 |
| E200 + G4S | GGS | |
| linker + IgG | | |
| hinge + G4S linker | | |
| N-term extended | DFPGHNYTTRNILPGLNITSEPKSSDKTHT | 50 |
| E200 + IgG hinge | | |
| N-term extended | DFPGHNYTTRNILPGLNITSGSEPKSSDKTHT | 51 |
| E200 + GS | | |
| linker + IgG hinge | | |
| N-term extended | DFPGHNYTTRNILPGLNITSGGGGSEPKSSDKTHT | 52 |
| E200 + G4S | | |
| linker + IgG hinge | | |
| N and C term | DFPGHNYTTRNILPGLNITSTFHKTSGSGKEPKSSDKTHT | 53 |
| extended | | |
| E200 + IgG hinge | | |
| N and C term | DFPGHNYTTRNILPGLNITSTFHKTSGSGKGSEPKSSDKTHT | 54 |
| extended | | |
| E200 + GS | | |
| linker + IgG hinge | | |
| N and C term | DFPGHNYTTRNILPGLNITSTFHKTSGSGKGGGGSEPKSSDKTH | 55 |
| extended E200 + | T | |
| G4S linker + IgG | | |
| hinge | | |
| N-term extended | DFPGHNYTTRNILPGLNITSEPKSSDKTHTGS | 56 |
| E200 + IgG hinge | | |
| + GS linker | | |
| N-term extended | DFPGHNYTTRNILPGLNITSGSEPKSSDKTHTGS | 57 |
| E200 + GS | | |
| linker + IgG hinge | | |
| + GS linker | | |
| N-term extended | DFPGHNYTTRNILPGLNITSGGGGSEPKSSDKTHTGS | 58 |
| E200 + G4S | | |
| linker + IgG hinge | | |
| + GS linker | | |
| N-term and C | DFPGHNYTTRNILPGLNITSTFHKTSGSGKEPKSSDKTHTGS | 59 |
| term extended | | |
| E200 + IgG | | |
| hinge + GS linker | | |
| N-term and C | DFPGHNYTTRNILPGLNITSTFHKTSGSGKGSEPKSSDKTHTGS | 60 |
| term extended | | |
| E200 + GS | | |
| linker + IgG | | |
| hinge + GS linker | | |
| N-term and C | DFPGHNYTTRNILPGLNITSTFHKTSGSGKGGGGSEPKSSDKTH | 61 |
| term extended | TGS | |
| E200 + G4S | | |
| linker + IgG | | |
| hinge + GS linker | | |
| N-term extended | DFPGHNYTTRNILPGLNITSEPKSSDKTHTGGGGS | 62 |
| E200 + IgG hinge | | |
| + G4S linker | | |
| N-term extended | DFPGHNYTTRNILPGLNITSGSEPKSSDKTHTGGGGS | 63 |
| E200 + GS | | |
| linker + IgG hinge | | |
| + G4S linker | | |
| N-term extended | DFPGHNYTTRNILPGLNITSGGGGSEPKSSDKTHTGGGGS | 64 |
| E200 + G4S | | |
| linker + IgG hinge | | |
| + G4S linker | | |
| N-term and C | DFPGHNYTTRNILPGLNITSTFHKTSGSGKEPKSSDKTHTGGGG | 65 |
| term extended | S | |
| E200 + IgG | | |
| hinge + G4S linker | | |
| N-term and C | DFPGHNYTTRNILPGLNITSTFHKTSGSGKGSEPKSSDKTHTGG | 66 |
| term extended | GGS | |
| E200 + GS | | |
| linker + IgG | | |
| hinge + G4S linker | | |
| N-term and C | DFPGHNYTTRNILPGLNITSTFHKTSGSGKGGGGSEPKSSDKTH | 67 |
| term extended | TGGGGS | |
| E200 + G4S | | |
| linker + IgG | | |
| hinge + G4S linker | | |
| IgG hinge + G4S | DKTHTSPPSPAPELLGGGGSDFPGHNYTTRNILPGLNITS | 68 |
| linker + N-term | | |
| extended E200 | | |
| (underlined) | | |
| G4S linker + IgG | GGGGSEPKSSDKTHTSPPSPAPELLGGGGSDFPGHNYTTRNILP | 69 |
| hinge + N-term | GLNITS | |
| extended E200 | | |
| (underlined) | | |
| G4S linker | GGGGS | 70 |
| IgG hinge region | EPKSSDKTHT | 71 |
| linker | | |
| GS linker + IgG | GSEPKSSDKTHTSPPSPAPELL | 72 |
| hinge | | |
| G4S linker + IgG | GGGGSEPKSSDKTHTSPPSPAPELLGGGGS | 73 |
| hinge + G4S | | |
| IgG hinge + GS | EPKSSDKTHTGS | 74 |
| linker | | |
| IgG hinge + G4S | EPKSSDKTHTGGGGS | 75 |
| linker | | |
| Exemplary C | TFHKT | 138 |
| terminal | | |
| extension of | | |
| E200 epitope | | |
| Exemplary N | DFP | 139 |
| terminal | | |
| extension of | | |
| E200 epitope | | |
| N terminal | DFPGHNYTTRNILPGLNITS | 140 |
| extended core E | | |
| 200 epitope | | |
| Exemplary IgG1 | EPKSCDKTHTSPPSPAP | 141 |
| hinge region for | | |
| monomeric | | |
| fusion proteins | | |
| (two C to S | | |
| mutations) | | |
| Exemplary IgG1 | EPKSSDKTHTSPPSPAP | 142 |
| hinge region for | | |
| monomeric | | |
| fusion proteins | | |
| (three C to S | | |
| mutations) | | |
| Exemplary G4S | GGGGS | 143 |
| linker sequence | | |
| Exemplary | LEVLFQGPVRR | 144 |
| cleavable linker | | |
| (protease | | |
| recognition site; | | |
| cleavage | | |
| between Q and | | |
| G residues) | | |
| Exemplary E200- | DFPGHNYTTRNILPGLNITSTFHKTSGSGKGGGGSEPKSCDKTH | 145 |
| Fc fusion | TSPPSPAPPVAGPSVFLFPPKPKDTLMISRTPEVTCVVVGVSHED | |
| sequence | PEVKFNWYVDGVEVHNAKTKPREEQYQSTYRVVSVLTVLHQDW | |
| “DetR1” | LNGKEYKCKVSNKQLPSPIEKTISKAKGQPREPQVYTLPPSRDEL | |
| (monomeric and | TKNQVSLTCLVKGFYPSDIAVEWESNGQPENNYKTTPPVLDSDG | |
| Fc attenuated | SFFLYSKLTVDKSRWQQGNVFSCSVMHEALHNHYTQKSLSLSP | |
| E233P; GK | | |
| L234V; L235A; | | |
| ΔG236, D265G, | | |
| N297Q, A327Q, | | |
| A330S shown in | | |
| underline and | | |
| italics) | | |
| (E200 moiety | | |
| underlined; linker | | |
| in bold) | | |
| Exemplary E200- | GHNYTTRNILPGLNITSTFHKTSGSGKGGGGSEPKSCDKTHTSP | 146 |
| sequence | PSPAPPVAGPSVFLFPPKPKDTLMISRTPEVTCVVVGVSHEDPE | |
| “DetR2” | VKFNWYVDGVEVHNAKTKPREEQYQSTYRVVSVLTVLHQDWLN | |
| (monomeric and | GKEYKCKVSNKQLPSPIEKTISKAKGQPREPQVYTLPPSRDELTK | |
| Fc attenuated | NQVSLTCLVKGFYPSDIAVEWESNGQPENNYKTTPPVLDSDGSF | |
| E233P; | FLYSKLTVDKSRWQQGNVFSCSVMHEALHNHYTQKSLSLSPGK | |
| L234V; L235A; | | |
| ΔG236, D265G, | | |
| N297Q, A327Q, | | |
| A330S) | | |
| (E200 moiety | | |
| underlined; linker | | |
| in bold; cys to | | |
| serine in italics; | | |
| Exemplary E200- | DFPGHNYTTRNILPGLNITSTFHKTSGSGKGGGGSEPKSSDKTH | 147 |
| Fc fusion | TSPPSPAPEAARGPSVFLFPPKPKDTLMISRTPEVTCVVVDVSHE | |
| sequence | DPEVKFNWYVDGVEVHNAKTKPREEQYNSTYRVVSVLTVLHQD | |
| “DetR1” | WLNGKEYKCKVSNKALPAPIEKTISKAKGQPREPQVYTLPPSRD | |
| + LALA + G236R | ELTKNQVSLTCLVKGFYPSDIAVEWESNGQPENNYKTTPPVLDS | |
| (monomeric and | DGSFFLYSKLTVDKSRWQQGNVFSCSVMHEALHNHYTQKSLSL | |
| Fc attenuated) | SPGK | |
| (E200 moiety | | |
| underlined; linker | | |
| in bold; cys to | | |
| serine in italics; | | |
| LALA + C1q | | |
| mutations in | | |
| italics and | | |
| underline) | | |
| Exemplary E200- | GHNYTTRNILPGLNITSTFHKTSGSGKGGGGSEPKSSDKTHTSP | 148 |
| Fc fusion | PSPAPEAARGPSVFLFPPKPKDTLMISRTPEVTCVVVDVSHEDP | |
| sequence | EVKFNWYVDGVEVHNAKTKPREEQYNSTYRVVSVLTVLHQDWL | |
| “DetR2” | NGKEYKCKVSNKALPAPIEKTISKAKGQPREPQVYTLPPSRDELT | |
| + LALA + G236R | KNQVSLTCLVKGFYPSDIAVEWESNGQPENNYKTTPPVLDSDGS | |
| (monomeric and | FFLYSKLTVDKSRWQQGNVFSCSVMHEALHNHYTQKSLSLSPG | |
| Fc attenuated) | K | |
| (E200 moiety | | |
| underlined; linker | | |
| in bold; cys to | | |
| serine in italics; | | |
| LALA + C1q | | |
| mutations in | | |
| italics and | | |
| underline) | | |
| Exemplary E200- | DFPGHNYTTRNILPGLNITSTFHKTSGSGKGGGGSEPKSCDKTH | 149 |
| Fc fusion sequence | TCPPCPAPPVAGPSVFLFPPKPKDTLMISRTPEVTCVVVGVSHE | |
| “DetR1” | DPEVKFNWYVDGVEVHNAKTKPREEQYQSTYRVVSVLTVLHQD | |
| (Fc attenuated - | WLNGKEYKCKVSNKQLPSPIEKTISKAKGQPREPQVYTLPPSRD | |
| dimeric) | ELTKNQVSLTCLVKGFYPSDIAVEWESNGQPENNYKTTPPVLDS | |
| (E200 moiety | DGSFFLYSKLTVDKSRWQQGNVFSCSVMHEALHNHYTQKSLSL | |
| underlined; linker | SPGK | |
| in bold) | | |
| Exemplary E200- | GHNYTTRNILPGLNITSTFHKTSGSGKGGGGSEPKSCDKTHTCP | 150 |
| Fc fusion | PCPAPPVAGPSVFLFPPKPKDTLMISRTPEVTCVVVGVSHEDPE | |
| sequence | VKFNWYVDGVEVHNAKTKPREEQYQSTYRVVSVLTVLHQDWLN | |
| “DetR2” | GKEYKCKVSNKQLPSPIEKTISKAKGQPREPQVYTLPPSRDELTK | |
| (dimeric Fc | NQVSLTCLVKGFYPSDIAVEWESNGQPENNYKTTPPVLDSDGSF | |
| attenuated) | FLYSKLTVDKSRWQQGNVFSCSVMHEALHNHYTQKSLSLSPGK | |
| (E200 moiety | | |
| underlined; linker | | |
| in bold) | | |
| Exemplary E200- | DFPGHNYTTRNILPGLNITSTFHKTSGSGKGGGGSEPKSSDKTH | 151 |
| Fc fusion | TCPPCPAPEAARGPSVFLFPPKPKDTLMISRTPEVTCVVVDVSH | |
| sequence | EDPEVKFNWYVDGVEVHNAKTKPREEQYNSTYRVVSVLTVLHQ | |
| “DetR1” | DWLNGKEYKCKVSNKALPAPIEKTISKAKGQPREPQVYTLPPSR | |
| + LALA + G236R | DELTKNQVSLTCLVKGFYPSDIAVEWESNGQPENNYKTTPPVLD | |
| (dimeric Fc | SDGSFFLYSKLTVDKSRWQQGNVFSCSVMHEALHNHYTQKSLS | |
| attenuated) | LSPGK | |
| (E200 moiety | | |
| underlined; linker | | |
| in bold; | | |
| LALA + C1q | | |
| mutations in | | |
| italics and | | |
| underline) | | |
| Exemplary E200- | GHNYTTRNILPGLNITSTFHKTSGSGKGGGGSEPKSSDKTHTCP | 152 |
| Fc fusion | PCPAPEAARGPSVFLFPPKPKDTLMISRTPEVTCVVVDVSHEDP | |
| sequence | EVKFNWYVDGVEVHNAKTKPREEQYNSTYRVVSVLTVLHQDWL | |
| “DetR2” | NGKEYKCKVSNKALPAPIEKTISKAKGQPREPQVYTLPPSRDELT | |
| + LALA + G236R | KNQVSLTCLVKGFYPSDIAVEWESNGQPENNYKTTPPVLDSDGS | |
| (dimeric Fc | FFLYSKLTVDKSRWQQGNVFSCSVMHEALHNHYTQKSLSLSPG | |
| attenuated) | K | |
| (E200 moiety | | |
| underlined; linker | | |
| in bold; | | |
| LALA + C1q | | |
| mutations in | | |
| italics and | | |
| underline) | | |
| Exemplary E200- | DFPGHNYTTRNILPGLNITSTFHKTSGSGKGGGGSLEVLFQGPV | 153 |
| Fc fusion | RREPKSSDKTHTSPPSPAPPVAGPSVFLFPPKPKDTLMISRTPEV | |
| sequence | TCVVVGVSHEDPEVKFNWYVDGVEVHNAKTKPREEQYQSTYRV | |
| “DetR1” | VSVLTVLHQDWLNGKEYKCKVSNKQLPSPIEKTISKAKGQPREP | |
| With cleavable | QVYTLPPSRDELTKNQVSLTCLVKGFYPSDIAVEWESNGQPENN | |
| sequence | YKTTPPVLDSDGSFFLYSKLTVDKSRWQQGNVFSCSVMHEALH | |
| between Fc | NHYTQKSLSLSPGK | |
| region and E200 | | |
| moiety | | |
| (monomeric Fc | | |
| attenuated) | | |
| (E200 moiety | | |
| underlined; linker | | |
| in bold; cleavable | | |
| linker in italics; | | |
| cys to serine | | |
| subs in italics) | | |
| Exemplary E200- | DFPGHNYTTRNILPGLNITSTFHKTSGSGKGGGGSLEVLFQGPV | 154 |
| sequence | RREPKSSDKTHTCPPCPAPPVAGPSVFLFPPKPKDTLMISRTPE | |
| Fc fusion | VTCVVVGVSHEDPEVKFNWYVDGVEVHNAKTKPREEQYQSTYR | |
| “DetR1” | VVSVLTVLHQDWLNGKEYKCKVSNKQLPSPIEKTISKAKGQPRE | |
| With cleavable | PQVYTLPPSRDELTKNQVSLTCLVKGFYPSDIAVEWESNGQPEN | |
| sequence | NYKTTPPVLDSDGSFFLYSKLTVDKSRWQQGNVFSCSVMHEAL | |
| between Fc | HNHYTQKSLSLSPGK | |
| region and E200 | | |
| moiety | | |
| (dimeric Fc | | |
| attenuated) | | |
| (E200 moiety | | |
| underlined; linker | | |
| in bold; cleavable | | |
| linker in italics) | | |
| Exemplary E200- | DFPGHNYTTRNILPGLNITSTFHKTSGSGKGGGGSEPKSSDKTH | 155 |
| Fc fusion sequence | TSPPSPAPEAARGPSVFLFPPKPKDTLMASRTPEVTCVVVDVSH | |
| “DetR1” + LALA + G236R: | EDPEVKFNWYVDGVEVHNAKTKPREEQYNSTYRVVSVLTVLAQ | |
| I253A/H310A | DWLNGKEYKCKVSNKALPAPIEKTISKAKGQPREPQVYTLPPSR | |
| (monomeric, Fc | DELTKNQVSLTCLVKGFYPSDIAVEWESNGQPENNYKTTPPVLD | |
| attenuated and | SDGSFFLYSKLTVDKSRWQQGNVFSCSVMHEALHNHYTQKSLS | |
| FcRN | LSPGK | |
| attenuated) | | |
| (E200 moiety | | |
| underlined; | | |
| linker in bold; | | |
| LALA + C1q | | |
| mutations in | | |
| italics and | | |
| underline; | | |
| cysteine | | |
| substitutions in | | |
| italics; | | |
| substitutions for | | |
| reducing FcRN | | |
| binding in bold | | |
| and underline) | | |
| Exemplary E200- | DFPGHNYTTRNILPGLNITSTFHKTSGSGKGGGGSEPKSSDKTH | 156 |
| Fc fusion sequence | TCPPCPAPEAARGPSVFLFPPKPKDTLMASRTPEVTCVVVDVSH | |
| “DetR1” | EDPEVKFNWYVDGVEVHNAKTKPREEQYNSTYRVVSVLTVLAQ | |
| + LALA + G236R: | DWLNGKEYKCKVSNKALPAPIEKTISKAKGQPREPQVYTLPPSR | |
| I253A/H310A | DELTKNQVSLTCLVKGFYPSDIAVEWESNGQPENNYKTTPPVLD | |
| (dimeric, Fc | SDGSFFLYSKLTVDKSRWQQGNVFSCSVMHEALHNHYTQKSLS | |
| attenuated and | LSPGK | |
| FcRN | | |
| attenuated) | | |
| (E200 moiety | | |
| underlined; linker | | |
| in bold; | | |
| LALA + C1q | | |
| mutations in | | |
| italics and | | |
| underline; | | |
| substitutions for | | |
| reducing FcRN | | |
| binding in bold | | |
| and underline) | | |
| Exemplary E200- | DFPGHNYTTRNILPGLNITSTFHKTSGSGKGGGGSEPKSSDKTH | 157 |
| Fc fusion | TCPPCPAPPVAGPSVFLFPPKPKDTLMISRTPEVTCVVVGVSHE | |
| sequence | DPEVKFNWYVDGVEVHNAKTKPREEQYQSTYRVVSVLTVLHQD | |
| “DetR1” | WLNGKEYKCKVSNKQLPSPIEKTISKAKGQPREPQVYTLPPSRD | |
| (Fc attenuated) | ELTKNQVSLTCLVKGFYPSDIAVEWESNGQPENNYKTTPPVLDS | |
| (E200 moiety | DGSFFLYSKLTVDKSRWQQGNVFSCSVMHEALHNHYTQKSLSL | |
| underlined; linker | SPGK | |
| in bold) | | |
| Exemplary E200- | DFPGHNYTTRNILPGLNITSTFHKTSGSGKGGGGSEPKSSDKTH | 158 |
| Fc fusion | TSPPSPAPEAAGGPSVFLFPPKPKDTLMISRTPEVTCVVVDVSH | |
| sequence | EDPEVKFNWYVDGVEVHNAKTKPREEQYNSTYRVVSVLTVLHQD | |
| “DetR1” | WLNGKEYKCKVSNKALGAPIEKTISKAKGQPREPQVYTLPPSRK | |
| (Fc attenuated) | ELTKNQVSLTCLVKGFYPSDIAVEWESNGQPENNYKTTPPVLKS | |
| (E200 moiety | DGSFFLYSKLTVDKSRWQQGNVFSCSVMHEALHNHYTQKSLSL | |
| underlined; linker | SPGK | |
| in bold) | | |
| “Knob” Fc region | | |
| (no dimerisation | | |
| due to C to S | | |
| substitutions in | | |
| hinge region | | |
| “Hole” Fc region | EPKSSDKTHTCPPCPAPEAAGGPSVFLFPPKPKDTLMISRTPEVT | 159 |
| (no E200 moiety) | CVVVDVSHEDPEVKFNWYVDGVEVHNAKTKPREEQYNSTYRVV | |
| | SVLTVLHQDWLNGKEYKCKVSNKALGAPIEKTISKAKGQPREPQ | |
| | VYTLPPSRDELTKNQVSLTCLVKGFYPSDIAVEWESNGQPENNY | |
| | DTTPPVLDSDGSFFLYSDLTVDKSRWQQGNVFSCSVMHEALHN | |
| | HYTQKSLSLSPGK | |
| Exemplary E200- | DFPGHNYTTRNILPGLNITSTFHKTSGSGKGGGGSEPKSSDKTH | 160 |
| Fc fusion | TCPPCPAPEAAGGPSVFLFPPKPKDTLMISRTPEVTCVVVDVSH | |
| sequence | EDPEVKFNWYVDGVEVHNAKTKPREEQYNSTYRVVSVLTVLHQ | |
| “DetR1” | DWLNGKEYKCKVSNKALGAPIEKTISKAKGQPREPQVYTLPPSR | |
| (Fc attenuated) | DELTKNQVSLTCLVKGFYPSDIAVEWESNGQPENNYDTTPPVLD | |
| (E200 moiety | SDGSFFLYSDLTVDKSRWQQGNVFSCSVMHEALHNHYTQKSLS | |
| underlined; linker | LSPGK | |
| in bold) | | |
| “Hole” Fc region | | |
| Exemplary E200- | DFPGHNYTTRNILPGLNITSTFHKTSGSGKGGGGSEPKSSDKTH | 161 |
| Fc fusion | TCPPCPAPEAAGGPSVFLFPPKPKDTLMISRTPEVTCVVVDVSH | |
| sequence | EDPEVKFNWYVDGVEVHNAKTKPREEQYNSTYRVVSVLTVLHQ | |
| “DetR1” | DWLNGKEYKCKVSNKALGAPIEKTISKAKGQPREPQVYTLPPSR | |
| (Fc attenuated) | DELTKNQVSLTCLVKGFYPSDIAVEWESNGQPENNYDTTPPVLD | |
| (E200 moiety | SDGSFFLYSDLTVDKSRWQQGNVFSCSVMHEALHNHYTQKSLS | |
| underlined; linker | LSPGK | |
| in bold) | | |
| “Hole” Fc region | | |
| (no dimerisation | | |
| due to C to S | | |
| substitutions in | | |
| hinge region | | |
| “Knob” Fc region | EPKSSDKTHTSPPSPAPEAAGGPSVFLEPPKPKDTLMISRTPEVT | 162 |
| (no E200 moiety) | CVVVDVSHEDPEVKFNWYVDGVEVHNAKTKPREEQYNSTYRVV | |
| | SVLTVLHQDWLNGKEYKCKVSNKALGAPIEKTISKAKGQPREPQ | |
| | VYTLPPSRKELTKNQVSLTCLVKGFYPSDIAVEWESNGQPENNY | |
| | KTTPPVLKSDGSFFLYSKLTVDKSRWQQGNVFSCSVMHEALHN | |
| | HYTQKSLSLSPGK | |

### BRIEF DESCRIPTION OF THE DRAWINGS

FIG. 1: Detection of untransduced T cells (UTD) (bottom panel) donor T cells expressing anti-nfP2X₇ CAR (middle panel) and T cells expressing anti-CD33 CAR (top panel).

FIG. 2: Flow cytometric analysis of untransduced T cells (UTD) (bottom panel), donor T cells expressing anti-nfP2X₇ CAR (middle panel) and Jurkat cells expressing anti-nfP2X₇ CAR (top panel) using three different biotinylated fusion proteins comprising an epitope of nfP2X₇ receptor. Fusion proteins used were: DetR1 dimeric (SEQ ID NO: 149); DetR1 monomer (SEQ ID NO: 158) and DetR2 monomer (SEQ ID NO: 146).

FIG. 3: Proportion of CD25+/CD69+ and PD-L1+ cells at 24 hours (A), 48 hours (B) and 72 hours (C) following detection with monomeric or dimeric fusion proteins (having the amino acid sequence of SEQ ID NOs: 158 and 149, respectively).

### DETAILED DESCRIPTION OF THE EMBODIMENTS

An important consideration during cellular immunotherapy protocols is the need to assess whether the CAR T cells continue to proliferate in vivo, following administration. This includes determining whether the cells continue to be present in the circulation of the patient at various time points following initial infusion of the cells. Although methods for detecting CAR T cells exist, these typically rely on modifications to the CAR or immune cells expressing the CAR (such as the inclusion of a fluorescent label or tag). This approach is less desirable as it requires further genetic modification of cells, and or the infusion of extraneous material to the subject.

The approach of the present invention is non-invasive, does not require modification of the CAR or immune cells expressing the CAR, and enables the rapid determination of the presence of genetically modified immune cells expressing a receptor for binding to dysfunctional P2X₇ receptor in a patient sample.

The present invention provides fusion proteins (including monomeric. homodimeric or heterodimeric molecules derived therefrom), comprising a linear epitope derived from the P2X₇ receptor (such as exemplified in any of SEQ ID NOs: 14 or 7) and an Fc region of an antibody. Such fusion proteins have particular use in the in vitro detection of CAR T cells, when coupled to a moiety for enabling detection of the protein.

In especially preferred embodiments, the Fc fusion proteins are designed so as to comprise only a single copy of the linear epitope derived from the P2X₇ receptor. This can be accomplished, as further described herein, by introducing amino acid substitutions into the Fc region to prevent homodimerisation, or alternatively, using the well-known knob-into-holes technology for ensuring formation of an asymmetric heterodimeric molecule (eg comprising a E200 peptide-Fc fusion protein and an Fc region that does not comprise an E200 peptide). Such monomeric or asymmetric heterodimeric molecules have the advantage of reducing activation of the target immune cells and preventing unwanted exhaustion of the target immune cells (as further described herein in the examples), particularly if there is a need or intention to determine the function or activation status of the CAR T cells following detection thereof. Without wishing to be bound by theory, the inventors believe that this is due to the reduced ability of the molecules to cross-link either two different CAR receptors on one cell or two different CAR receptors on two separate CAR expressing cells.

### Definitions

Unless defined otherwise, technical and scientific terms used herein have the same meaning as commonly understood by one of ordinary skill in the art to which this invention belongs.

For purposes of interpreting this specification, the following definitions will generally apply and whenever appropriate, terms used in the singular will also include the plural and vice versa.

As used herein, the term “and/or”, e.g., “X and/or Y” will be understood to mean either “X and Y” or “X or Y” and shall be taken to provide explicit support for both meanings or for either meaning.

The articles “a” and “an” are used herein to refer to one or to more than one (i.e. to at least one) of the grammatical object of the article. By way of example, “a dysfunctional P2X₇ receptor epitope moiety” means one dysfunctional P2X₇ receptor epitope moiety or more than one dysfunctional P2X₇ receptor epitope moiety.

As used herein, except where the context requires otherwise, the term “comprise” and variations of the term, such as “comprising”, “comprises” and “comprised”, are not intended to exclude further additives, components, integers or steps.

“Purinergic receptor” generally refers to a receptor that uses a purine (such as ATP) as a ligand.

“P2X₇ receptor” generally refers to a purinergic receptor formed from three protein subunits or monomers, with at least one of the monomers having an amino acid sequence substantially as shown in SEQ ID NO: 1 in Table 1 herein.

To the extent that P2X₇ receptor is formed from three monomers, it is a “trimer” or “trimeric”. “P2X₇ receptor” encompasses naturally occurring variants of P2X₇ receptor, e.g., wherein the P2X₇ monomers are splice variants, allelic variants, SNPs and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO: 1. In certain embodiments the P2X₇ receptor may have an amino acid sequence that is modified, for example various of the amino acids in the sequence shown in SEQ ID NO: 1 may be substituted, deleted, or a residue may be inserted.

“Functional P2X₇ receptor” generally refers to a form of the P2X₇ receptor having three intact binding sites or clefts for binding to ATP. When bound to ATP, the functional receptor forms a non-selective sodium/calcium channel that converts to a pore-like structure that enables the ingress of calcium ions and molecules of up to 900 Da into the cytosol, one consequence of which may be induction of programmed cell death. In normal homeostasis, expression of functional P2X₇ receptors is generally limited to cells that undergo programmed cell death such as thymocytes, dendritic cells, lymphocytes, macrophages and monocytes. There may also be some expression of functional P2X₇ receptors on erythrocytes and other cell types.

“Dysfunctional P2X₇ receptor” (also called “non-functional” or (nf) P2X₇) is a P2X₇ receptor that has an impaired response to ATP such that it is unable to form an apoptotic pore under physiological conditions. A dysfunctional P2X₇ receptor or (nfP2X₇ receptor) generally refers to a form of a P2X₇ receptor having a conformation, distinct from functional P2X₇, whereby the receptor is unable to form an apoptotic pore, but which is still able to operate as a non-selective channel through the maintenance of a single functional ATP binding site located between adjacent monomers. One example arises where one or more of the monomers has a cis isomerisation at Pro210 (according to SEQ ID NO: 1). The isomerisation may arise from any molecular event that leads to misfolding of the monomer, including for example, mutation of monomer primary sequence or abnormal post translational processing. One consequence of the isomerisation is that the receptor is unable to bind to ATP at one, or more particularly two, ATP binding sites on the trimer and as a consequence not be able to extend the opening of the channel. In the circumstances, the receptor cannot form a pore and this limits the extent to which calcium ions may enter the cytosol. Dysfunctional P2X₇ receptors are expressed on a wide range of epithelial and haematopoietic cancers. As used herein, the term “dysfunctional P2X₇ receptors” may be used interchangeably with the term “non-functional P2X₇ receptors” or “nfP2X₇ receptors”.

“Cancer associated-P2X₇ receptors” are generally P2X₇ receptors that are found on cancer cells (including, pre-neoplastic, neoplastic, malignant, benign or metastatic cells), but not on non-cancer or normal cells.

“E200 epitope” generally refers to an epitope having the sequence GHNYTTRNILPGLNITC (SEQ ID NO: 2). Variants thereof are exemplified in Table 1 and include any of SEQ ID NOs: 3, or 7 to 69 and 122.

“E300 epitope” generally refers to an epitope having the sequence KYYKENNVEKRTLIK (SEQ ID NO: 4) or a variant thereof, as defined in SEQ ID NO: 5.

A “composite epitope” generally refers to an epitope that is formed from the juxtaposition of the E200 and E300 epitopes or parts of these epitopes. An example of a composite epitope comprising E200 and E300 epitopes is GHNYTTRNILPGAGAKYYKENNVEK (SEQ ID NO: 6).

As used herein, the term “antigen” is intended to include substances that bind to or evoke the production of one or more antibodies and may comprise, but is not limited to, proteins, peptides, polypeptides, oligopeptides, lipids, carbohydrates, and combinations thereof, for example a glycosylated protein or a glycolipid. The term “antigen” as used herein refers to a molecular entity that may be expressed on a target cell and that can be recognised by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

The terms “binds to”, “specifically binds to” or “specific for” with respect to a receptor referring to an antigen-binding domain that recognises and binds a dysfunctional P2X₇ receptor, is intended to mean that the receptor does not substantially recognise or bind to other antigens in a sample.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

The terms “immune cell” or “immune effector cell” refer to a cell that may be part of the immune system and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells. The cells may be naturally occurring or generated by cytokine exposure, artificial/genetically modified cells (such as iPSCs and other artificial cell types). Preferred immune cells are cells with cytotoxic effector function such as alpha-beta T cells, NK cells, NKT cells, ILC, CIK cells, LAK cells or gamma-delta T cells. “Effector function” means a specialised function of a cell, e.g. in a T cell an effector function may be cytolytic activity or helper cell activity including the secretion of cytokines.

The term “autologous” as used herein refers to any material derived from the same subject to whom it is later re-introduced.

The term “allogeneic” as used herein refers to any material derived from a different subject of the same species as the subject to whom the material is re-introduced.

The terms “engineered cell” and “genetically modified cell” as used herein can be used interchangeably. The terms mean containing and/or expressing a foreign gene or nucleic acid sequence that in turn modifies the genotype or phenotype of the cell or its progeny. Especially, the terms refer to the fact that cells, preferentially immune cells, can be manipulated by recombinant methods well known in the art to express stably or transiently peptides or proteins that are not expressed in these cells in the natural state. For example, immune cells are engineered to express an artificial construct such as a chimeric antigen receptor on their cell surface. For example, the CAR sequences may be delivered into cells using an adenoviral, adeno-associated viral (AAV)-based, retroviral or lentiviral vector or any other pseudotyped variations thereof or any other gene delivery mechanism such as electroporation or lipofection with CRISPR/Cas9, transposons (e.g. sleeping-beauty) or variations thereof. The gene delivery may be in the form of mRNA (transient) or DNA (transient or permanent).

Amino acid structure and single and three letter abbreviations used throughout the specification are defined in Table 2, which lists the twenty proteinogenic naturally occurring amino acids which occur in proteins as L-isomers.

TABLE 2
(1)

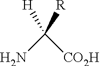

(2)

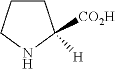

| | Three-letter | One-letter | Structure of side chain |
|---|---|---|---|
| Amino Acid | Abbreviation | symbol | (R) |
| Alanine | Ala | A | —CH₃ |
| Arginine | Arg | R | —(CH₂)₃NHC(═N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

As used herein, the term “non-proteinogenic amino acid” refers to an amino acid having a side chain that does not occur in the naturally occurring L-α-amino acids recited in Table 2. Examples of non-proteinogenic amino acids and derivatives include, but are not limited to, norleucine, 4-aminobutyric acid, 4-amino-3-hydroxy-5-phenylpentanoic acid, 6-aminohexanoic acid, t-butylglycine, norvaline, phenylglycine, ornithine, citrulline, sarcosine, 4-amino-3-hydroxy-6-methylheptanoic acid, 2-thienyl alanine and/or D-isomers of natural amino acids

As used herein, the term “α-amino acid” refers to an amino acid that has a single carbon atom (the α-carbon atom) separating a carboxyl terminus (C-terminus) and an amino terminus (N-terminus). An α-amino acid includes naturally occurring and non-naturally occurring L-amino acids and their D-isomers and derivatives thereof such as salts or derivatives where functional groups are protected by suitable protecting groups. Unless otherwise stated, the term “amino acid” as used herein refers to an α-amino acid.

The term “alkyl” refers to a straight chain or branched saturated hydrocarbon group having 1 to 6 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, and 5-methylpentyl.

As used herein, the term “subject” refers to a mammal such as mouse, rat, cow, pig, goat, chicken, dog, monkey or human. Preferentially, the subject is a human. The subject may be a subject suffering from a disorder such as cancer (a patient). As used herein, the terms “subject” and “individual” may be used interchangeably.

### Dysfunctional P2X₇ Receptor Epitope Moiety

The present invention provides fusion proteins comprising a dysfunctional P2X₇ receptor epitope moiety.

The dysfunctional P2X₇ receptor epitope moiety may be provided in the form of a dysfunctional P2X₇ receptor, or a fragment of a dysfunctional P2X₇ receptor, that has at least one of the three ATP binding sites that are formed at the interface between adjacent correctly packed monomers that are unable to bind ATP. Such receptors are unable to extend the opening of the non-selective calcium channels to apoptotic pores.

In accordance with the present invention, the dysfunctional P2X₇ receptor epitope moiety is typically in the form of a peptide fragment of a dysfunctional P2X₇ receptor. Typically, the peptide comprises an epitope that is not found or not available for binding on a functional P2X₇ receptor.

In some embodiments, the peptide comprises the proline at amino acid position 210 of the dysfunctional P2X₇ receptor. In some embodiments, the peptide comprises one or more amino acid residues spanning from glycine at amino acid position 200 to cysteine at amino acid position 216, inclusive, of the dysfunctional P2X₇ receptor.

A range of peptide fragments of a dysfunctional P2X₇ receptor are known and discussed in PCT/AU2002/000061 (and in corresponding publications WO 2002/057306 and U.S. Pat. Nos. 7,326,415, 7,888,473, 7,531,171, 8,080,635, 8,399,617, 8,709,425, 9,663,584, or U.S. Pat. No. 10,450,380), PCT/AU2008/001364 (and in corresponding publications WO 2009/033233 and U.S. Pat. Nos. 8,440,186, 9,181,320, 9,944,701 or 10,597,45) and PCT/AU2009/000869 (and in corresponding publications WO 2010/000041 and U.S. Pat. No. 8,597,643, 9,328,155 or 10,238,716) the contents of all of which are incorporated in entirety. Exemplary peptides within these specifications which include epitopes contemplated for use in this invention are described below.

| PCT publication | Peptide sequence |
|---|---|
| WO 2002/057306 | GHNYTTRNILPGLNIT (SEQ ID NO: 3) |
| WO 2002/057306 | GHNYTTRNILPGLNITC |
| | (SEQ ID NO: 2) (also referred to |
| | herein as the “E200” epitope) |
| WO 2009/033233 | KYYKENNVEKRTLIKVF (SEQ ID NO: 4) |
| | (also referred to |
| | herein as the “E300” epitope) |
| WO 2010/000041 | GHNYTTRNILPGAGAKYYKENNVEK |
| | (SEQ ID NO: 6) (also |
| | referred to herein as the |
| | “E200/E300” or “composite” |
| | epitope) |

Non-limiting examples of variations of the E200 peptide sequence (including with N and/or C terminal extensions, and various linker, hinge or spacer regions) are provided in Table 1.

The amino acid sequences of any one of SEQ ID NOs: 2 to 7 may comprise a portion of the epitope moiety that is recognised or capable of being bound by a receptor expressed on an immune cell (also referred to herein as the “recognition sequence” of the epitope moiety).

In some embodiments, the epitope moiety comprises or consists of an amino acid sequence selected from any of the peptide sequences listed in Table 1 above.

In some embodiments, the N-terminus of the epitope moiety is a free amine (—NH₂).

In some embodiments, the C-terminus of the epitope moiety is a free acid (—COOH). In some embodiments, the C-terminus is a derivative or analogue of a free acid group, for example an ester (—COOC1-6alkyl) or a primary or secondary amide (—CONHR4 wherein R4 is selected from H and C1-6alkyl). Advantageously, having a C-terminus that is a derivative or analogue of a free acid group may improve the biological stability of the peptide compared to the free acid. In some embodiments, the C-terminus is a derivative or analogue of a free acid group that comprises a functional moiety, for example biotin.

In any embodiment of the first or second aspects, the epitope of a dysfunctional P2X₇ receptor, comprises or consists of an epitope that is only found on dysfunctional P2X₇ receptor but is not found on a functional form of the P2X₇ receptor. In other words, preferably, the polypeptide comprises or consists of an epitope that is specific to a dysfunctional P2X₇ receptor.

In further embodiments of the first or second aspects, the fusion protein comprises an epitope corresponding to the E200, E300 or composite E200/E300 epitopes as herein defined. It will be within the purview of the skilled person to obtain various polypeptides for use in accordance with the invention. For example the skilled person will appreciate that it is possible to include additional amino acids N- or C-terminal to the region comprising the epitope bound by the anti-nfP2X₇ receptor CAR. In a non-limiting example, and in the context of E200, which is typically defined as having an amino acid sequence substantially as defined in SEQ ID NO: 2 or 7 (and having a minimum sequence as defined in SEQ ID NO: 14), additional amino acids derived from the native sequence of P2X₇ receptor can be included in the polypeptide, for example, the residues “DFP” N-terminal to the epitope in the P2X₇ receptor sequence and/or residues “TFHKT” C-terminal to the epitope in the P2X₇ receptor sequence. In any embodiment, the polypeptide may comprise at least 1, at least 2, at least 3, at least 4, at least 5 or at least 6 amino acids derived from the P2X₇ receptor sequence, in addition to the sequence of the E200 or E300 or composite epitopes.

In a preferred embodiment, the sequence of the E200 epitope is further modified to substitute the cysteine residue (residue 17 in SEQ ID NO: 2) to a serine residue (eg to provide the sequence of SEQ ID NO: 7). The skilled person will appreciate that this can be done to reduce likelihood of any disulphide bonding between the polypeptide and another molecule.

It will also be within the purview of the skilled person to include additional amino acid residues to the E200, E300 or composite epitopes (or extended epitopes as discussed in the paragraph above), such as, for example, by the addition of at least 1, at least 2, at least 3, at least 4, at least 5, at least 6, at least 7, at least 8, at least 9 or at least 10 additional amino acid residues to the N- and C-terminal regions of peptides consisting of the amino acid sequence of the relevant epitope. Typically such additional amino acids can be derived from linker sequences (such as peptides comprising glycine and serine residues); or be derived from the hinge region of an immunoglobulin. Typically no more than 30, no more than 25, or no more than 20 amino acid residues are added to the N- and/or C-terminal residues of the E200, E300 or composite epitopes as defined herein.

### Fc Region

In any embodiment, the amino acid sequence of the epitope of a dysfunctional P2X₇ receptor may be fused via its C terminal region to the N terminal region of an Fc region of an antibody, or variant thereof. In any embodiment, the amino acid sequence of epitope of a dysfunctional P2X₇ receptor may be fused via its N terminal region to the C terminal region of an Fc region of an antibody, or variant thereof.

Preferably, the Fc region of the fusion protein comprises two heavy chain fragments, more preferably the CH2 and CH3 domains of said heavy chain.

The Fc region may comprise one or more amino acid sequence modifications compared to naturally occurring Fc sequences. The Fc region may comprise one or more amino acid substitutions, such as substitution of one or more cysteine residues, so as to prevent dimerisation of the molecule to identical molecules. It will be appreciated that any amino acid substitution which prevents dimerisation of the Fc regions may be employed. As such, in vivo, the Fc fusion proteins described herein may be monomeric proteins.

The Fc region of the fusion protein may therefore comprise one or more amino acid substitutions compared to naturally occurring Fc sequences, which prevent or reduce the ability of the Fc region to homodimerise. Preferably, the amino acid substitutions comprise one or more substitutions of the cysteine residues so as to prevent the formation of disulphide bonds between Fc molecules. The cysteine residues of the Fc region may be substituted to any other amino acid residue, optionally to glycine, serine, alanine, lysine and glutamic acid, preferably glycine or serine.

The cysteine residues for substitution are preferably one or more of the cysteine residues located in the region of the Fc region which corresponds to the hinge region of an immunoglobulin. Examples of the IgG1 hinge regions, and variations thereof including cysteine to serine substitutions are provided herein in Table 3. The hinge region of an immunoglobulin (eg of IgG1) comprises three cysteine residues (which are number C220, C226 and C229 according to EU numbering. Accordingly, in any embodiment, at least one, at least two, or all three of the cysteine residues in the immunoglobulin hinge region are substituted. Preferably, at least two or all three of the cysteine residues are substituted. More preferably, all cysteine residues in the Fc region, such as the hinge region, are substituted. In particularly preferred embodiments, at least one of C226 and C229 are substituted, preferably wherein both C226 and C229 are substituted.

Accordingly, in preferred embodiments, the fusion protein comprises a hinge region for linking the a dysfunctional P2X₇ receptor epitope moiety and Fc region of an antibody, wherein the hinge region comprises an amino acid sequence that corresponds to any of the sequences set forth in SEQ ID NOs: 76 to 113, or 136 to 137 or 141 or 142.

In further embodiments, the fusion protein region may comprise an Fc region corresponding to an Fc “hole” or “knob” for use in a “knob-in-hole” heterodimer. The use of such Fc sequences is known in the art and provides for an asymmetric heterodimeric molecule comprising a fusion protein with a single copy of the epitope moiety as described herein and Fc region, bound to a further Fc region that does not comprise the epitope moiety.

The skilled person will be familiar with technology and Fc sequences for enabling the formation of so-called monomeric fusion proteins, including although not limited to the use of the “knobs-into-holes” IgG1 format (Ridgway et al., (1996), Protein Eng, 9:617-621). Such approaches in the context of the present invention, enable expression and purification of a heterodimeric fusion protein with only one copy of the peptide epitope (eg an epitope moiety derived from the E200 epitope as herein described), per molecule. Examples of the “knob-into-hole” Fc pairing is provided herein in SEQ ID NOs: 157 and 159 (knob and hole, respectively), 158 and 159, respectively, 160 and 162 (hole and knob, respectively) and 161 and 162, respectively. Accordingly, in any embodiment, the present invention provides a fusion protein comprising the amino acid sequence of any of SEQ ID NOs: 2 to 69 and 122, linked to an Fc region as defined in SEQ ID NO: 160 or 162, wherein the fusion protein is capable of forming a heterodimer with an Fc region that does not comprise an E200 peptide moiety.

As such, a fusion protein of the invention is preferably one that is capable of forming a heterodimeric molecule that comprises a single E200-containing amino acid sequence. (In other words, the Fc portion of the fusion protein may form a heterodimer with an Fc region of an antibody that does not comprise an E200 peptide fused thereto).

Preferably, the Fc region comprises one or more substitutions for ablating or reducing effector function, such as to reduce binding and activation via the FcR as further described below.

The term “Fc region” herein is used to define a C-terminal region of an immunoglobulin heavy chain that contains at least a portion of the constant region. In other words, the Fc region contains two heavy chain fragments comprising the CH2 and CH3 domains of an antibody. In the context of the present invention, the Fc region comprises two heavy chain fragments, preferably the CH2 and CH3 domains of said heavy chain. The two heavy chain fragments are held together by two or more disulfide bonds and by hydrophobic interactions of the CH3 domains. The heavy chain constant domains that correspond to the different classes of immunoglobulins are called a, 8, ¿, Y, and u, respectively.

In some aspects, the fusion protein does not exhibit any effector function or any detectable effector function. “Effector functions” or “effector activities” refer to those biological activities attributable to the Fc region of an antibody, which vary with the antibody isotype. Examples of antibody effector functions include: C1q binding and complement dependent cytotoxicity (CDC); Fc receptor binding; antibody dependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96° non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006); WO 2013/120929 A1).

In preferred embodiments, the Fc fusion proteins of the invention comprise Fc regions with reduced effector function. Fc regions with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831). Further, alterations may be made in the Fc region that result in altered (i.e., diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000) (eg G236R).

Further examples of modified Fc regions include those comprising the “LALALS” (amino acid substitutions L234A/L235A/M428L/N434S as described in Zalevsky et al., (2010) Nat. Biotechnol. 28:157-159); the LALAPG (L234A/L235A/P329G amino acid substitutions as described in Gunn et al., (2021, Immunity 54:815).

In any embodiment, the Fc region of the Fc fusion proteins of the invention may comprise at least the “LALA” mutations (L234A and L235A) for reducing binding to FcR. The fusion protein may in addition or alternatively comprise the mutation G346R for abrogating recruitment of complement C1q.

Other Fc modifications for use in the present invention include variants that reduce or ablate binding to FcγRs and/or complement proteins, thereby reducing or ablating Fc-mediated effector functions such as ADCC, ADCP, and CDC. Such variants are also referred to herein as “knockout variants” or “KO variants”. Variants that reduce binding to FcγRs and complement are useful for reducing unwanted interactions mediated by the Fc region. Preferred knockout variants are described in US 2008-0242845 A1, published on Oct. 2, 2008, entitled “Fc Variants with Optimized Properties, expressly incorporated by reference herein. Preferred modifications include but are not limited substitutions, insertions, and deletions at positions 234, 235, 236, 237, 267, 269, 325, and 328, wherein numbering is according to the EU index. Preferred substitutions include but are not limited to 234G, 235G, 236R, 237K, 267R, 269R, 325L, and 328R, wherein numbering is according to the EU index. A preferred variant comprises 236R/328R. Variants may be used in the context of any IgG isotype or IgG isotype Fc region, including but not limited to human IgG1, IgG2, IgG3, and/or IgG4. Preferred IgG Fc regions for reducing FcγR and complement binding and reducing Fc-mediated effector functions are IgG2 and IgG4 Fc regions. Hybrid isotypes may also be useful, for example hybrid IgG1/IgG2 isotypes as described in U.S. Ser. No. 11/256,060. Other modifications for reducing FcγR and complement interactions include but are not limited to substitutions 297A, 297D, 234A, 235A, 237A, 318A, 228P, 236E, AG236, 265G, 268Q, 297Q, 309L, 330S, 331S, 327Q, 220S, 226S, 229S, 238S, 233P, 234A, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691, incorporated by reference in its entirety.

In some aspects, the Fc region of the fusion protein includes mutations to the complement (C1q) and/or to Fc gamma receptor (FcγR) binding sites. In some aspects, such mutations can render the fusion protein incapable of antibody directed cytotoxicity (ADCC) and complement directed cytotoxicity (CDC).

The Fc region as used in the context of the present invention preferably does not trigger cytotoxicity such as antibody-dependent cellular cytotoxicity (ADCC) or complement dependent cytotoxicity (CDC).

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an anti-inflammatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

### Linker Region Between the Dysfunctional P2X₇ Receptor Epitope and Fc Region

The dysfunctional P2X₇ receptor epitope and Fc region of an antibody may be joined directly or via a linker sequence. The linker sequence may be a spacer sequence as herein defined or as exemplified in Table 1 or 3. Alternatively, the linker sequence may be any amino acid based linker sequence commonly in use in the field.

A linker is usually a peptide having a length of up to 20 amino acids although may be up to 50 amino acids in length. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 or more amino acids. For example, the herein provided fusion protein may comprise a linker between the epitope of a dysfunctional P2X₇ receptor and the Fc region of the antibody, such as between the N-terminus of the Fc regions and the C-terminus of dysfunctional P2X₇ receptor epitope. As another example, the herein provided fusion protein may comprise a linker between the dysfunctional P2X₇ receptor epitope and the Fc region of the antibody, such as between the C-terminus of the Fc regions and the N-terminus of the dysfunctional P2X₇ receptor epitope moiety. Particularly, the dysfunctional P2X₇ receptor epitope moiety may be fused via a linker at the C-terminus to the N-terminus of the Fc region. Such linkers have the advantage that they can make it more likely that the different polypeptides of the fusion protein fold independently and behave as expected. Thus, in the context of the present invention the dysfunctional P2X₇ receptor epitope moiety and the Fc region of an antibody may be comprised in a single-chain multi-functional polypeptide.

In some aspects, the fusion protein of the present invention includes a peptide linker. In some aspects, the peptide linker links a dysfunctional P2X₇ receptor epitope moiety with an Fc region of an antibody. In some aspects, the peptide linker can include the amino acid sequence Gly-Gly-Ser (GGS), Gly-Gly-Gly-Ser (GGGS) or Gly-Gly-Gly-Gly-Ser (GGGGS). In some aspects, the peptide linker can include the amino acid sequence GGGGS (a linker of 6 amino acids in length) or even longer. The linker may a series of repeating glycine and serine residues (GS) of different lengths, i.e., (GS) n where n is any number from 1 to 15 or more. For example, the linker may be (GS) 3 (i.e., GSGSGS) or longer (GS) 11 or longer. It will be appreciated that n can be any number including 1, 2, 3, 4, 5, 6, 7, 8, 9, 10, 11 or more. Fusion proteins having linkers of such length are included within the scope of the present invention. Preferably n is no more than 3 (ie such that when n equals 3 the linker is GSGSGS).

In further embodiments, the linker may comprise inclusion of an amino acid that provides rigidity, such as lysine. For example, in certain embodiments, the linker region may also comprise the sequence GSGK.

The peptide linker may consist of a series of repeats of Thr-Pro (TP) comprising one or more additional amino acids N and C terminal to the repeat sequence. For example, the linker may comprise or consist of the sequence GTPTPTPTPTGEF (also known as the TP5 linker). In further aspects, the linker may be a short and/or alpha-helical rigid linker (e.g. A (EAAAK) 3A, PAPAP or a dipeptide such as LE or CC).

In further embodiments, as an alternative or in addition to a glycine-serine-based linker region as described above, the fusion protein may comprise a dysfunctional P2X₇ receptor epitope moiety, linked to an Fc region of an antibody, via a hinge region. The linking between the dysfunctional P2X₇ receptor epitope moiety and the Fc region may comprise a combination of hinge region and linker regions.

Examples of suitable hinge regions include hinge regions derived from immunoglobulins. The hinge region may be derived from an IgG1, IgG2, IgG3 or IgG4, and may comprise one or more amino acid substitutions, (for example to prevent or reduce the likelihood of disulphide bridge formation). Alternative hinge sequences may be derived from alternative immunoglobulin domains, CD8A, CD8B, CD4 or CD28, TRAC, TRBC, TRGC, TRDC.

Further linker sequences may also include any sequence of any length of CL/CH1 domain but not all residues of CL/CH1 domain; for example the first 5-12 amino acid residues of the CL/CH1 domains. Linkers can be derived from immunoglobulin heavy chains of any isotype, including for example Cy1, Cy2, Cy3, Cy4, Ca1, Ca2, Co, CE, and Cu. Linkers can be derived from immunoglobulin light chain, for example CK or CA. Linker sequences may also be derived from other proteins such as Ig-like proteins (e.g. TCR, FcR, KIR), hinge region-derived sequences, and other natural sequences from other proteins.

Table 3 below provides non-limiting examples of suitable hinge regions for use in joining the dysfunctional P2X₇ receptor epitope moiety and Fc regions, in the molecules of the invention.

It will be appreciated that the dysfunctional P2X₇ receptor epitope moiety may be joined to the Fc regions by more than one linker and/or more than one hinge region. For example, the fusion protein may comprise (N to C terminus), the dysfunctional P2X₇ receptor epitope moiety, conjugated directly to the Fc region. Alternatively, the fusion protein may comprise the dysfunctional P2X₇ receptor epitope moiety, followed by a linker region, then the Fc region. Further still, the fusion protein may comprise the dysfunctional P2X₇ receptor epitope moiety, followed by a linker region, then a hinge region, and then the Fc region. In a further embodiment still, the fusion protein may comprise the dysfunctional P2X₇ receptor epitope moiety, followed by a linker region, then a hinge region, a further linker region, then the Fc region. Of course, the skilled person will appreciate that the alternative configuration is possible (ie wherein the dysfunctional P2X₇ receptor epitope moiety is joined to the C terminus of the Fc region, via one or more linker and/or hinge regions.

TABLE 3
further exemplary linker/hinge
region sequences

| | SEQ | |
|---|---|---|
| Amino acid | ID | |
| sequence | NO: | Description |
| EPKSSDKTHTSPPSP | 76 | Sequences derived |
| EPKSSDKTHTSPP | 77 | from IgG1 (ie |
| EPKSSDKTHTSPPP | 78 | modified IgG1 |
| EPKSSDKTHT | 79 | hinge regions) |
| DKTHTSPPSP | 80 | |
| DKTHTSPP | 81 | |
| EPKSDKTHTPPP | 82 | |
| EPKSDKTHTSPPSP | 83 | |
| EPKSGDKTHTGPPGP | 84 | |
| EPKSGDKTHTGPP | 85 | |
| EPKSGDKTHTGPPP | 86 | |
| EPKSGDKTHT | 87 | |
| DKTHTGPPGP | 88 | |
| DKTHTGPP | 89 | |
| EPKSDKTHTGPPGP | 90 | |
| EPKSGDKTHTSPPSP | 91 | |
| EPKSGDKTHTGPPSP | 92 | |
| EPKSSDKTHTGPPGP | 93 | |
| EPKSSDKTHTGPPSP | 94 | |
| EPKSSDKTHTGPP | 95 | |
| EPKSGDKTHTSPP | 96 | |
| EPKSTDKTHTTPPTP | 97 | |
| EPKSTDKTHTTPP | 98 | |
| EPKSTDKTHTTPPP | 99 | |
| EPKSTDKTHT | 100 | |
| DKTHTTPPTP | 101 | |
| DKTHTTPP | 102 | |
| EPKSDKTHTTPPTP | 103 | |
| EPKSSDKTHTTPPTP | 104 | |
| EPKSSDKTHTSPPTP | 105 | |
| EPKSADKTHTLPPMP | 106 | |
| EPKSVDKTHTLPPIP | 107 | |
| EPKSLDKTHTAPPAP | 108 | |
| EPKSVDKTHTAPP | 109 | |
| EPKSIDKTHTLPP | 110 | |
| DKTHTAPPLP | 111 | |
| DKTHTVPPLP | 112 | |
| EPKSSDKTHTSP | 113 | |
| EPKSCDKTHTCPPCP | 114 | Wild type IgG1 |
| ERKCCVECPPCP | 115 | Mutated IgG2 |
| ERKXXVEXPPXP | 116 | Mutated IgG2 |
| ERKXXVEXPP | 117 | Mutated IgG2 |
| VEXPPXP | 118 | Mutated IgG2 |
| ELKTPLGDTTHTCPRCP | 119 | Wild-type IgG3 |
| ELKTPLGDTTHTXPRXP | 120 | Mutated IgG3 |
| ELKTPLGDTTHTXPR | 12 | Mutated IgG3 |
| ELKTPLGDTTHT | 123 | Mutated IgG3 |
| EPKSCDTPPPCPRCP | 124 | Wild-type IgG3 |
| EPKSXDTPPPXPRXP | 125 | Mutated IgG3 |
| EPKSXDTPPPXPR | 126 | Mutated IgG3 |
| EPKSXDTPPP | 127 | Mutated IgG3 |
| DTPPPXPRXP | 128 | Mutated IgG3 |
| ESKYGPPCPSCP | 129 | Wild-type IgG4 |
| EXKYGPPCPXCP | 130 | Mutated IgG4 |
| EXKYGPPCP | 131 | Mutated IgG4 |
| KYGPPCPXCP | 132 | Mutated IgG4 |
| EPKSCDKTHTCP | 133 | Wild type IgG1 (v2) |
| EPKSCDTPPPCP | 134 | Wild type IgG3 (v2) |
| ESKYGPPSPSSP | 135 | Mutated IgG4 |
| ERKSSVESPPSP | 136 | Mutated IgG2 |
| EPKSSDTPPPSP | 137 | Mutated IgG3 |

In certain embodiments, the dysfunctional P2X₇ receptor epitope moiety is directly fused to the Fc region of an antibody, such that there is no linker between the two regions of the fusion protein.

In certain embodiments, the dysfunctional P2X₇ receptor epitope moiety is joined to the Fc region of an antibody, via a cleavable linker.

Cleavable linkers are well known in the art, and include for example, the sequence defined in SEQ ID NO: 144 and which defines a cleavage site for Human Rhinovirus 3C protease. Proteases for sue in cleaving such cleavage sites are also readily available from commercial providers (eg: Pierce HRV 3C Protease.

Other known cleavable linkers and other linker which may be used in accordance with the present invention are disclosed in Chen et al., (2013) Adv. Drug. Deliv. Rev. 65:1357-1369; the contents of which are incorporated herein by reference.

In further embodiments of the second aspect of the invention, the dysfunctional P2X₇ receptor epitope moiety is joined to the further amino acid sequence of the polypeptide via a spacer comprising a polysaccharide having at least 15 carbon atoms selected from the group consisting of dextrans, pullulans, inulins, amylose, cellulose, hemicelluloses, xylan, glucomannan, pectin, chitosan and chitin.

In further embodiments still of the second aspect of the invention, the polypeptide comprises a dysfunctional P2X₇ receptor epitope moiety joined to the modification for enabling detection of the polypeptide via a spacer comprising a polysaccharide having at least 15 carbon atoms selected from the group consisting of dextrans, pullulans, inulins, amylose, cellulose, hemicelluloses, xylan, glucomannan, pectin, chitosan and chitin.

The spacer unit of the polypeptide for use according to the second aspect of the invention preferably comprises at least 15 carbon atoms, and is preferable selected from the group consisting of oligopeptides, polyethylene glycols, enzymatically degradable units or affinity units, which provide a cleavable, non-covalent connection of detection moiety and the dysfunctional P2X₇ receptor epitope moiety.

Suitable oligopeptides comprise at least 2 amino acids, preferable up to 10 amino acids. Most preferred are oligopeptides having a sequence of amino acids of GGGSK. Preferable polyethylene glycols (PEG) comprise 10-200 ethylene glycol units.

As enzymatically degradable spacer, any molecule which can be cleaved by a specific enzyme can be used. Suitable as enzymatically degradable spacer S are, for example, polysaccharides, proteins, peptides, depsipeptides, polyesters, nucleic acids, and derivatives thereof which can be cleaved by hydrolases. The enzymatically degradable spacer can be composed of more than one different enzymatically degradable units, which are degradable by the same or different enzyme.

Preferred polysaccharides are, for example, dextrans, pullulans, inulins, amylose, cellulose, hemicelluloses, such as xylan or glucomannan, pectin, chitosan, or chitin.

The detection moiety and the dysfunctional P2X₇ receptor epitope moiety can be covalently or non-covalently coupled to the spacer. Methods for covalently or non-covalently conjugation are known by persons skilled in the art. In case of a covalent bound between the detection moiety and/or dysfunctional P2X₇ receptor epitope moiety and the spacer, a direct reaction of an activated group either on the detection moiety and/or dysfunctional P2X₇ receptor epitope moiety or on the spacer with an functional group on either the spacer or on the detection moiety and/or dysfunctional P2X₇ receptor epitope moiety or via an heterobifunctional linker molecule, which is firstly reacted with one and secondly reacted with the other binding partner is possible.

For a non-covalent or quasi-covalent coupling of detection moiety D to dysfunctional P2X₇ receptor epitope moiety via spacer, the spacer may be provided with an affinity unit which be cleaved by a release agent when required. Affinity units comprise for example biotin, avidin and/or streptavidin resulting in a quasi-covalent binding having dissociation constants of less than 10-9 M.

The term “release agent” refers to any compound capable of binding to a part of the affinity unit. For example, a biotin-avidin affinity unit used as spacer may be cleaved by streptavidin or adding an access of free biotin. By competitive reaction, the affinity unit is cleaved thereby releasing detection moiety from the dysfunctional P2X₇ receptor epitope moiety (or vice versa). Suitable affinity unit and release agents are disclosed by James Hirsch et al. in Analytical Biochemistry 308 (2002) 343-357.

The affinity unit can be provided with a detection means, i.e. possess a label that can be used for detection. The detection means may be the same or different that those disrobed described for detection moiety. The use of the affinity unit provided with a detection means as spacer allows further quantification of the CAR cells.

### Receptors and Immune Cells Expressing Same

The present invention finds application in methods for detecting subpopulations of immune cells that express receptors for binding to dysfunctional P2X₇ receptor. The receptor is preferably a chimeric antigen receptor (CAR) or variant thereof. The receptor may also be a modified TCR.

In general, a CAR, variant thereof, or TCR, may comprise an extracellular domain (extracellular part) comprising the antigen binding domain, a transmembrane domain and an intracellular signaling domain. The extracellular domain may be linked to the transmembrane domain by a linker. The extracellular domain may also comprise a signal peptide. Preferably, the extracellular part of the CAR, variant thereof, or TCR comprises an nfP2X₇ binding domain that recognises the E200 (or E300 or E200-300 composite) epitope as disclosed herein.

Typically, the antigen-recognition domain of the CAR or TCR includes a binding polypeptide that includes amino acid sequence homology to one or more complementarity determining regions (CDRs) of an antibody that binds to a dysfunctional P2X₇ receptor. In any embodiment, the binding polypeptide includes amino acid sequence homology to the CDR1, 2 and 3 domains of the VH and/or VL chain of an antibody that binds to a dysfunctional P2X₇ receptor. As will be appreciated, the CAR will preferably be able to recognise the same dysfunctional P2X₇ receptor epitope moiety that is present on the fusion proteins of the invention.

Although it will be appreciated that any CAR for binding to dysfunctional P2X₇ receptor can be used in accordance with the method of the invention, in preferred embodiments, the binding polypeptide of the CAR comprises the amino acid sequence of the CDRs of the VH and/or VL chain of an antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding US patent U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. Nos. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In further embodiments, the binding polypeptide of the CAR comprises the amino acid sequence of the VH and/or VL chains of an antibody described in PCT/AU2002/000061 or PCT/AU2002/001204 (or in any one of the corresponding US patents U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding US patent U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. Nos. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In further embodiments still, the binding polypeptide of the CAR comprises the amino acid sequence of an antibody or fragment thereof described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding US patent U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. Nos. 8,597,643, 9,328,155 or 10,238,716), PCT/AU2010/001070 (or in any one of the corresponding publications WO/2011/020155, U.S. Pat. Nos. 9,127,059, 9,688,771, or 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

The CAR typically also comprises a signal peptide. A “signal peptide” refers to a peptide sequence that directs the transport and localisation of the protein within a cell, e.g. to a certain cell organelle (such as the endoplasmic reticulum) and/or the cell surface.

Generally, an “antigen binding domain” (or antigen recognition domain) refers to the region of the CAR that specifically binds to an antigen (and thereby is able to target a cell containing the antigen). A CAR may comprise one or more antigen binding domains. Generally, the targeting regions on the CAR are extracellular. The antigen binding domain may comprise an antibody or an antibody binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in an scFv the variable regions of an immunoglobulin heavy chain and light chain are fused by a flexible linker to form a scFv. Such a linker may be for example the “(G₄/S₁)₃-linker” and variations thereof but the skilled person will appreciate that various linker sequences and formats may be used.

CARs may also comprise a “hinge” region (sometimes called a spacer region or linker region) joining the antigen binding domain to the transmembrane domain. This is typically a hydrophilic region that is between the antigen binding domain and the transmembrane domain. A CAR may comprise an extracellular hinge domain but it is also possible to leave out such a hinge. The hinge region may include for example, Fc fragments of antibodies or fragments thereof, hinge regions of antibodies or fragments thereof, CH2 or CH3 regions of antibodies, accessory proteins, artificial hinge sequences or combinations thereof. One example of a hinge region is the CD8alpha hinge.

The transmembrane domain of the CAR may be derived from any desired natural or synthetic source for such a domain. When the source is natural, the domain may be derived from any membrane-bound or transmembrane protein. The transmembrane domain may be derived for example from CD8alpha or CD28. When the key signalling and antigen recognition modules (domains) are on two (or even more) polypeptides, then the CAR may have two (or more) transmembrane domains. The splitting of key signalling and antigen recognition modules enables small molecule-dependent, titratable and reversible control over CAR cell expression (Wu et al, 2015, Science 350:293-303) due to small molecule-dependent heterodimerising domains in each polypeptide of the CAR.

The cytoplasmic domain (or the intracellular signaling domain) of the CAR is responsible for activation of at least one of the normal effector functions of the immune cell in which the CAR is expressed. “Effector function” means a specialised function of a cell, e.g. in a T cell an effector function may be cytolytic activity or helper cell activity including the secretion of cytokines. The intracellular signalling domain refers to the part of a protein that transduces the effector function signal and directs the cell expressing the CAR to perform a specialised function. The intracellular signalling domain may include any complete, mutated or truncated part of the intracellular signalling domain of a given protein sufficient to transduce a signal that initiates or blocks immune cell effector functions.

The function of the intracellular domains may be pro- or anti-inflammatory and/or immunomodulatory, or a combination of such.

Examples of intracellular signalling domains for use in the CARs include the cytoplasmic signaling sequences of the T cell receptor (TCR) and co-receptors that initiate signal transduction following antigen receptor engagement.

Primary cytoplasmic signalling sequences that act in a stimulatory manner may contain ITAMs (immunoreceptor tyrosine-based activation motifs) signalling motifs.

Examples of ITAM containing primary cytoplasmic signalling sequences often used in CARs are those derived from TCR zeta (CD3 zeta), FcR gamma, FcR beta, CD3 gamma, CD3 delta, CD3 epsilon, CD5, CD22, CD79a, CD79b and CD66d. Most prominent is the sequence derived from CD3 zeta.

The cytoplasmic domain of the CAR may be designed to comprise the CD3-zeta signaling domain by itself or combined with any other desired cytoplasmic domain(s). The cytoplasmic domain of the CAR can comprise a CD3 zeta chain portion and a co-stimulatory signalling region. The co-stimulatory signalling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C and B7-H3.

In some embodiments, the activation receptor (from which a portion of signalling domain is derived) is the CD3 co-receptor complex or is an Fc receptor.

In some embodiments, the co-stimulatory receptor (from which a portion of signalling domain is derived) is selected from the group consisting of CD27, CD28, CD-30, CD40, DAP10, OX40, 4-1BB (CD137) and ICOS.

In some embodiments, the co-stimulatory receptor (from which a portion of signalling domain is derived) is selected from the group consisting of CD28, OX40 or 4-1BB.

The cytoplasmic signalling sequences within the cytoplasmic signalling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signalling domain of CD3-zeta and the signalling domain of CD28. In another example the cytoplasmic domain may comprise the signalling domain of CD3-zeta and the signalling domain of CD27. In a further example, the cytoplasmic domain may comprise the signalling domain of CD3-zeta, the signalling domain of CD28, and the signalling domain of CD27.

As aforementioned, either the extracellular part or the transmembrane domain or the cytoplasmic domain of a CAR may also comprise a heterodimerising domain for the aim of splitting key signalling and antigen recognition modules of the CAR.

The CAR which binds to a radiolabeled molecule of an invention, e.g., a CAR comprising an nfP2X₇ E200 binding domain, may be designed to comprise any portion or part of the above-mentioned domains as described herein in any order and/or combination resulting in a functional CAR.

The affinity at which the dysfunctional P2X₇ receptor binding domain of the CAR binds to the nfP2X₇ recognition site E200 of a radiolabeled molecule of the invention can vary, but generally the binding affinity may be in the range of approximately 100 UM, approximately 10 μM, approximately 1 μM, approximately 100 nM, approximately 10 nM, or approximately 1 nM, preferably at least about 10 pM or 1 pM. In preferred embodiments, the binding affinity is at least about 1 nM or at least about 10 nM.

The receptor (such as a CAR, variant thereof, or TCR, or variant thereof) is typically expressed by an immune cell.

The immune cell may be an “engineered cell”, “genetically modified cell”, or “immune effector cell” as described herein. Further, the immune cell may be an immune cell precursor that is capable of differentiating into an immune cell. A cell that is capable of differentiating into an immune cell (e.g. T cell that will express the dysfunctional P2X₇ CAR) may be a stem cell, multi-lineage progenitor cell or induced pluripotent stem.

The immune cell may be a leukocyte, a Peripheral Blood Mononuclear Cell (PBMC), a lymphocyte, a T cell (including a CD4+ T cell or a CD8+ T cell), a natural killer cell, a natural killer T cell, or a γδ T cell.

In any embodiment, the immune cell may be a T cell, wherein optionally said T cell does not express TcRαβ, PD1, CD3 or CD96 (e.g. by way of knocking down or knocking out one of these genes on a genetic level or functional level).

In any embodiment, the immune cell optionally does not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out one of these genes on a genetic level or functional level).

In some embodiments, the immune cell includes two or more different receptors (e.g., two or more CARs, or variants thereof). The CARs may bind to different epitopes on the same target molecule (e.g., different epitopes on dysfunctional P2X₇ receptor). Alternatively, the CARs may bind different target molecules, such that only one of the CARs binds to dysfunctional P2X₇ receptors.

As used herein, the term “different CARS” or “different chimeric antigen receptors” refers to any two or more CARs that have either non-identical antigen-recognition and/or non-identical signalling domains. In one example, “different CARs” includes two CARs with the same antigen-recognition domains (e.g. both CARs may recognise a dysfunctional P2X₇ receptor), but have different signalling domains, such as one CAR having a signalling domain with a portion of an activation receptor and the other CAR having a signalling domain with a portion of an co-stimulatory receptor. As will be understood, at least one of the two or more CARs within this embodiment will have an antigen-recognition domain that recognises the dysfunctional P2X₇ receptor and the other CAR(s) may take any suitable form and may be directed against any suitable antigen.

### Methods for Detecting Immune Cells

It will be well within the purview of the skilled person to confirm the ability of a given fusion protein or polypeptide, as defined herein to be bound by the relevant receptor. For example in the context of nfP2X₇ receptor-binding CARs, the skilled person will be able to use routine techniques to confirm binding of the polypeptide (or series of polypeptides) by the CAR, and thereby determine the suitability of the polypeptide for use in methods of the invention.

In any embodiment, the cells in the biological sample which are complexed with a polypeptide as described herein are not reintroduced back into the individual.

It will be understood that the methods of the invention can be used to detect immune cells that express a receptor (including a CAR), which comprises an antigen binding domain that recognizes the epitope of the dysfunctional P2X₇ receptor contained on the fusion protein. The method and the fusion proteins/polypeptide of the invention enable detection of target cells that bind to the fusion protein/polypeptide via a modification on the fusion protein or polypeptide for enabling detection thereof (also referred to herein as the “detection moiety”.

In any embodiment of the second aspect of the invention, the biological sample may be any patient sample comprising the immune cells desired to be detected. Preferably, the sample is representative of the levels of circulating immune cells expressing a receptor binding to dysfunctional P2X₇ receptor. Preferably, the sample is a sample of peripheral blood, such as EDTA-anticoagulated peripheral blood, or derivative thereof, such as PBMCs (buffy coat).

It will be appreciated that the biological sample may be derived from tissue or other fluid in the body which comprises immune cells. Thus, the sample may also be derived from solid tissue which has been homogenised to generate a single cell suspension (eg using the gentleMACS Dissociator).

Moreover, upon contacting the fusion protein or polypeptide of the invention with any population of cells comprising the target cells (ie cells capable of binding to the dysfunctional P2X₇ receptor epitope on the fusion protein), the skilled person can make use of routine laboratory techniques to detect the fusion protein/cell complex, and thereby confirm the presence or absence of the immune cells in the sample.

Further still, the methods of the invention enable the quantification of immune cells expressing the receptor for binding to dysfunctional P2X₇ receptor.

It will be appreciated that the fusion protein or polypeptide for use according to the invention may comprise any detection moiety, possessing a property or function which can be used for direct and indirect detection purposes such as those selected from the group consisting of chromophore moiety, fluorescent moiety, phosphorescent moiety, luminescent moiety, light absorbing moiety, radioactive moiety, and chemically detectable moieties like haptens, e.g. biotin, avidin, streptavidin and derivates thereof or magnetic particles.

In preferred embodiments, the detection moiety is a fluorochrome, a magnetic particle or biotin.

Suitable fluorescent moieties are those known from the art of immunofluorescence technologies, e.g. flow cytometry or fluorescence microscopy. In these embodiments of the invention, the target cells labeled with the reagent are detected by exciting the detection moiety D and detecting the resulting emission (photoluminescence). In this embodiment, the detection moiety D is preferable a fluorescent moiety.

Useful fluorescent moieties might be protein-based, such as phycobiliproteins, polymeric, such as polyfluorenes, small organic molecule dyes, such as xanthenes, like fluorescein, or rhodamines, cyanines, oxazines, coumarins, acridines, oxadiazoles, pyrenes, pyrromethenes, or metallo-organic complexes, such as Ru, Eu, Pt complexes.

In one embodiment, the modification for enabling detection, especially a fluorochrome can be destroyed by oxidation in photo- or chemical bleaching procedures (U.S. Pat. No. 7,741,045 B2, EP 0810 428 B1 or DE10143757) such that the fluorescence is quenched.

Magnetic particles useful for enabling detection are preferably nano- to microscale magnetic particle, also known in the art as magnetic beads. The mean diameter of the beads can range from 10 nm to 10 μm. Biocompatible magnetic particles are commercially available and consist of, for example, forms of magnetically iron oxide coated by a shell of dextran molecules or silica. The solid support may also be polymers containing magnetic materials. Suitable particles are commercial available from Miltenyi Biotec GmbH, Germany under the trade name “MicroBeads” and “MACSiBeads”.

The cells that bind to the fusion protein or polypeptide comprising the modification for enabling detection, can be detected by fluorescence emission, by applying a magnetic field or by chemical reaction of the chemically detectable moiety.

In one embodiment of the invention, the detection moiety is a fluorescent moiety. Target cells labeled with fluorochrome-conjugate are detected by exciting the fluorescent moiety and analyzing the resulting fluorescence signal. The wavelength of the excitation is usually selected according to the absorption maximum of the fluorescent moiety and provided by LASER or LED sources as known in the art. If several different detection moieties are used for multiple color/parameter detection, care should be taken to select fluorescent moieties having not overlapping absorption spectra, at least not overlapping absorption maxima. In case of fluorescent moieties as detection moiety the targets may be detected, e.g., under a fluorescence microscope, in a flow cytometer, a spectrofluorometer, or a fluorescence scanner. Light emitted by chemiluminescence can be detected by similar instrumentation omitting the excitation.

In another embodiment of the invention, the detection moiety is a light absorbing moiety, which is detected by the difference between the irradiation light intensity and the transmitted or reflected light intensity. Light absorbing moieties might also be detected by photoacoustic imaging, which uses the absorption of a pulsed laser beam to generate an acoustic like an ultrasonic signal.

Radioactive detection moieties are detected though the radiation emitted by the radioactive isotopes. Suitable instrumentation for detection of radioactive radiation includes, for example, scintillation counters. In case of beta emission electron microscopy can also be used for detection.

Transition metal isotope mass tag moieties are detected by mass spectrometric methods such as ICP-MS, which is integrated in mass cytometry instrumentation. Metal labelling may also be useful for enabling detection and quantification of cells using CyTOF (cytometry by time of flight) methodologies. CyTOF allows the quantification of multiple cellular components simultaneously using an ICP-MS detector. For such applications, the fusion protein or polypeptide may be labelled with a lanthanide group of elements, and can be joined to the fusion protein or polypeptide via isotope polymers comprising diethylenetriaminepentaacetic acid (DTPA) chelator, for example. This enables thiol or maleimide links to Fc regions of the fusion protein or antibody via reduced disulfide bonds. Four to five polymers are bound to an antibody, resulting in about 100 isotope atoms per antibody. Tagged fusion proteins may be in solution, conjugated to beads, or surface immobilized. The cell staining follows the same procedures as in fluorescent staining for flow cytometry.

In the case of an Fc fusion protein that retains the ability to dimerise, complexes of the fusion protein and cells may be purified using Protein A beads, and the cells then quantified using conventional flow cytometry techniques.

Alternatively, a fluorophore-labelled anti-Fc antibody may be used to bind to and detect the presence of the fusion protein complex.

In the case where the Fc fusion protein is biotinylated, an anti-biotin antibody, optionally one that is fluorescently labelled, may be used to isolate the complex and detect the same.

Of course, in situations where the Fc fusion protein comprises a fluorescent label, the degree of fluorescence can be directly determined as a means for detecting the presence of the cells, and quantity thereof in the sample.

### EXAMPLES

### Example 1: Flow Cytometric Detection of CAR T Cells for Binding to Dysfunctional P2X₇ Receptor

Whole blood samples are treated for 10 min with 2 ml of NH₄Cl-based erythrocyte lysing solution (Beckman Coulter, Krefeld, Germany) and are washed with PBS, containing 0.5% HSA. After removal of the supernatant, cells are resuspended and 100 μl are transferred to a new flow cytometry tube. Following 15 min of incubation with a fusion protein comprising the sequence of SEQ ID NO: 158 or 146 (DetR1 and DetR2, respectively) cells are washed and incubated for 15 min with an anti-biotin antibody (Miltenyi Biotec, Bergisch Gladbach, Germany), 7-AAD, CD3-APC, and CD45-KrO (all purchased from Beckman Coulter Immunotech, Marseille, France).

After a final washing step, cells are acquired on a NAVIOS flow cytometer (Beckman Coulter, Krefeld, Germany). Cellular debris are excluded based on light scatter properties and CAR T cells are defined as 7-AAD-/CD45+/mononuclear cells/CD3+/CD19 CAR+.

### Example 2: Detection of CAR T Cells Using Exemplary Biotinylated Fusion Protein of the Invention

PBMCs were obtained from a donor and enriched for CD4+ and CD8+ T cells. The cells were then either left untransduced, or were transduced with lentiviral constructs encoding CARs using standard techniques. The constructs used also included a sequence encoding EGFR to enabling indirect detection of successfully transduced cells.

Untransduced T cells (UTD), and T cells expressing either an anti-nfP2X₇ CAR, or an anti-CD33 CAR (based on Lintuzumab) were stained with primary labelled anti-EGFR antibody cetuximab to detect truncated EGFR which was co-expressed downstream of the CAR receptor after a ribosomal skip (T2A) site.

The UTD cells are negative for both the CAR and the marker gene. For labelling of cetuximab, AF647 from ThermoFisher (Alexa Fluor™ 647 Antibody Labeling Kit, Catalog number: A20186) was used according to manufacturer instructions. The staining was done at 1 μg/mL.

The detection of T cells expressing anti-nfP2X₇ CAR was done using DetR1-Fc attenuated fusion protein (SEQ ID NO: 145), that was biotinylated using the NHS-LC-LC-Biotin biotinylation kit from ThermoFisher (EZ-Link™ NHS-LC-LC-Biotin Catalog number: 21343) according to manufacturer instructions. As a secondary antibody anti-Biotin antibody from Miltenyi Biotec (130-113-857, Biotin Antibody, VioBlue®) was used according to manufacturer instructions.

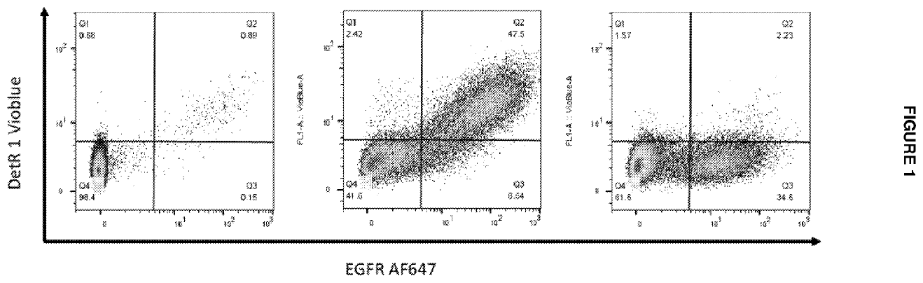

As shown in FIG. 1, the UTD and anti-CD33 CAR T cells were both negative after staining with the DetR1 biotinylated molecule, whereas the cells expressing anti-nfP2X₇ CAR showed double positive staining for the CAR positive cells: 1st via the marker gene truncated EGFR and 2nd via the CAR receptor itself using the Fc fusion protein. Thus, the detection reagent DetR1 (SEQ ID NO: 158) can be used to specifically identify anti-nfP2X₇ CAR expressing cells.

Similar experiments were performed using PBMCs obtained from two other individuals. The results (not shown) were similar to the above.

### Example 3: Detection of CAR T Cells Using Exemplary Monomeric and Dimeric Biotinylated Fusion Protein of the Invention

A similar experiment to that described in Example 2 was performed. T cells were obtained from a donor and either untransduced T cells (UTD) (bottom panel) or transduced with lentiviral constructs encoding anti-nfP2X₇ CAR (middle panel). For comparison, Jurkat cells were also transduced with the same lentiviral construct.

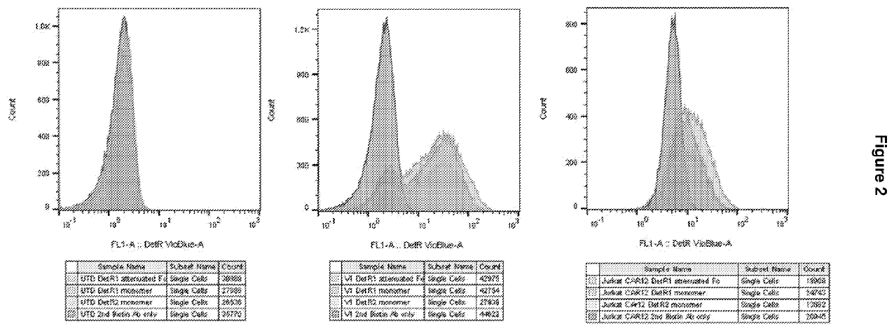

Cells expressing the CAR were detected using three different biotinylated fusion proteins comprising an epitope of nfP2X₇ receptor. The fusion proteins used were: DetR1 dimeric (SEQ ID NO: 149); DetR1 monomer (SEQ ID NO: 158) and DetR2 monomer (SEQ ID NO: 146). The results shown in FIG. 2 show that either monomeric or dimeric fusion proteins can be used to detect CAR-expressing cells. However, monomeric fusion proteins are preferable because they do not lead to cross-linking.

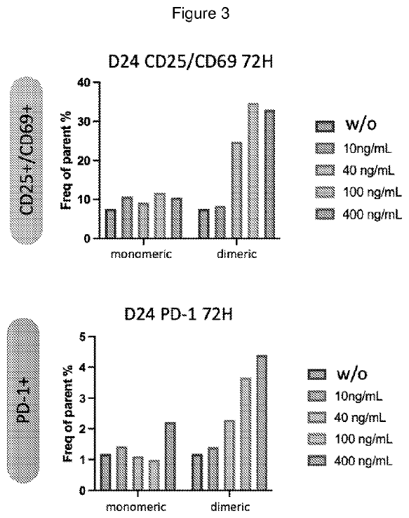

FIG. 3 shows, for example, differences in the expression of activation markers (CD25 and CD69) and exhaustion marker PD-1 in CAR T cells that have been contacted with either a monomeric fusion protein or a homodimeric fusion protein (eg comprising two copies of the E200 epitope sequence) as described herein. The results indicate that contacting CAR T cells using a monomeric detection reagent (such as having the amino acid sequence of SEQ ID NO: 158) leads to significantly less activation and exhaustion of the CAR T cells over a 72 hour period, compared to a homodimeric detection reagent (such as having the amino acid sequence of SEQ ID NO: 149). Thus, the use of a monomeric detection reagent (eg having only one copy of the E200 peptide for binding to the CAR) provides for particular advantages in relation to functional assessment of the CAR T cells subsequent to detection thereof. For example, if it is desirable to determine the function of the CAR T cells following detection, it would be preferable that the detection reagent has not lead to unwanted activation or exhaustion of the CAR T cells.

Similar experiments are conducted using heterodimeric asymmetric molecules as described herein (eg such that the molecules comprise dimerisation between an E200 peptide-Fc fusion protein and a non-identical Fc region of an antibody; using KIH technology). The results similarly show that contacting CAR T cells using a heterodimeric asymmetric molecule comprising a single copy of the E200 peptide sequence, leads to significantly less T cell activation and significantly less T cell exhaustion, in a concentration dependent manner, compared to when using a dimeric fusion protein that comprises two copies of the E200 peptide (eg wherein the dimer is a homodimer of E200-Fc fusion proteins). These results indicate that for the purposes of detecting CAR T cells (particularly if there is an intention to subsequently determine function or performing other in vitro analyses), it is preferable to use an asymmetric heterodimer molecule or monomeric fusion protein (ie comprising a single E200 peptide sequence) in order to minimise unwanted activation and exhaustion of the CAR T cells in the patient.

It will be understood that the invention disclosed and defined in this specification extends to all alternative combinations of two or more of the individual features mentioned or evident from the text or drawings. All of these different combinations constitute various alternative aspects of the invention.

1. A fusion protein comprising:
(i) a dysfunctional P2X₇ receptor epitope moiety; and
(ii) an Fc region of an antibody, wherein the Fc region has a reduced affinity for an Fc receptor compared to wild-type or naturally occurring Fc regions.

2. A fusion protein of claim 1, wherein the Fc region is an Fc region of an IgG, IgA, IgD, IgE, or IgM.

3. The fusion protein of claim 1 or 2, wherein the Fc region is from an IgG antibody, such as an IgG1, an IgG2, an IgG2b, an IgG3 or an IgG4 antibody.

4. The fusion protein of any one of claims 1 to 3, wherein the Fc region of the fusion protein comprises two heavy chain fragments, more preferably the CH2 and CH3 domains of said heavy chain.

5. The fusion protein of any one of claims 1 to 3, wherein the Fc region comprises one or more amino acid substitutions for reducing affinity for one or more of FcγRI, FcγRII and FcγRIII, and thereby reduce the ability of the fusion protein to elicit antibody-dependent cell-mediated toxicity (ADCC).

6. The fusion protein of any one of claims 1 to 5, wherein the fusion protein has an affinity for FcR that is less than about 250 nM, preferably less than 500 nM, less than 1000 nM, most preferably less than 2000 nM.

7. The fusion protein of any one of claims 1 to 6, wherein the Fc region of the fusion protein comprises one or more amino acid substitutions compared to naturally occurring Fc sequences, which prevent or reduce the ability of the Fc region to homodimerise.

8. The fusion protein of claim 7, wherein the amino acid substitutions comprise one or more substitutions of the cysteine residues so as to prevent the formation of disulphide bonds between Fc molecules.

9. The fusion protein of claim 8, wherein the one or more substitutions of the cysteine residues are to glycine, serine, alanine, lysine or glutamic acid, preferably glycine or serine.

10. The fusion protein of any one of claims 1 to 9, wherein the dysfunctional P2X₇ receptor epitope moiety comprises the amino acid sequence of an epitope which is found on dysfunctional P2X₇ receptor but not on functional P2X₇ receptor.

11. The fusion protein of any one of claims 1 to 10, wherein the amino acid sequence of the dysfunctional P2X₇ receptor epitope moiety comprises or consists at least of the amino acid sequence as set forth in SEQ ID NO: 14.

12. The fusion protein of any one of claims 1 to 11, wherein the amino acid sequence of the dysfunctional P2X₇ receptor epitope moiety comprises or consists at least of the amino acid sequence as set forth in SEQ ID NO: 7 or 9 or 122.

13. The fusion protein of any one of claims 1 to 11, wherein the amino acid sequence of the dysfunctional P2X₇ receptor epitope moiety comprises or consists at least of the amino acid sequence as set forth in any of SEQ ID NOs: 7 to 69, or 122, or sequences at least 80%, at least 81%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto, provided that the sequences comprise at least the sequence set forth in SEQ ID NO: 14 or 7.

14. The fusion protein of any one of claims 1 to 12, wherein the fusion protein comprises one or more modifications for enabling the detection of the fusion protein or complexes comprising the fusion protein.

15. The fusion protein of claim 14, wherein the one or more modifications to the fusion protein may be selected from: a fluorescent moiety, a metallic label (such as a lanthanide element) a magnetic particle, a chromophore moiety, a phosphorescent moiety, a luminescent moiety, a light absorbing moiety, a radioactive moiety, and chemically detectable moieties like haptens, e.g. biotin, avidin, streptavidin and derivatives thereof.

16. Use of a fusion protein of any one of claims 1 to 15, in a method for detecting genetically modified immune cells which express a receptor that comprises an antigen binding domain for binding to dysfunctional P2X₇ receptor.

17. The use of claim 16, wherein the immune cells are chimeric antigen receptor (CAR) cells.

18. The use of claim 17, wherein the use is for determination of the presence of the immune cells in a complex mixture, such as a biological sample obtained from a patient who previously received treatment with the immune cells.

19. An in vitro method for detecting an immune cell expressing a receptor that comprises an antigen binding domain for binding to dysfunctional P2X₇ receptor, the method comprising:
(i) providing a biological sample from a patient who has received a treatment with immune cells, preferably immune effector cells, wherein the cells express a receptor comprising an antigen binding domain for binding to dysfunctional P2X₇ receptor;
(ii) contacting the sample with a polypeptide, wherein the polypeptide comprises an epitope of dysfunctional P2X₇ receptor which is recognised by the antigen binding domain of the receptor, and wherein the polypeptide comprises a detection moiety for enabling detection of the polypeptide;
to thereby allow formation of a complex of the polypeptide bound to the cells;
(iii) detecting the complex,
thereby detecting an immune cell expressing a receptor having an antigen binding domain for binding to dysfunctional P2X₇ receptor.

20. An in vitro method for detecting immune cells expressing a chimeric antigen receptor (CAR) for binding to dysfunctional P2X₇ receptor, the method comprising:
(i) providing a biological sample from a patient who has received a treatment with immune cells, preferably immune effector cells, comprising a chimeric antigen receptor (CAR) for binding to dysfunctional P2X₇ receptor;
(ii) contacting the sample with a polypeptide, wherein the polypeptide comprises an epitope of dysfunctional P2X₇ receptor which is recognised by the CAR, and wherein the polypeptide comprises a detection moiety for enabling detection of the polypeptide;
to thereby allow formation of a complex of the polypeptide bound to the cells;
(iii) detecting the complex,
thereby detecting immune cells expressing a chimeric antigen receptor (CAR) for binding to dysfunctional P2X₇ receptor.

21. The method of claim 19 or 20 wherein the method comprises the step of first isolating the complex prior to the step of detecting.

22. The method of any one of claims 19 to 21, wherein the polypeptide comprises the amino acid sequence of a fusion protein of as defined in any of claims 1 to 15.

23. The method of any one of claims 19 to 21, wherein the polypeptide comprises a first portion comprising an epitope of dysfunctional P2X₇ receptor joined to a further moiety for facilitating the solubility and stability of the first portion.

24. The method of claim 23, wherein the further moiety is an amino acid sequence joined to the dysfunctional P2X₇ receptor epitope such as a linker or hinge region, such as an amino acid sequence as exemplified in Tables 1 and 3; or a spacer comprising a polysaccharide having at least 15 carbon atoms selected from the group consisting of dextrans, pullulans, inulins, amylose, cellulose, hemicelluloses, xylan, glucomannan, pectin, chitosan and chitin.

25. The method of claim 23, wherein the polypeptide is in the form of a fusion protein comprising an epitope of dysfunctional P2X₇ receptor joined to a further amino acid sequence selected from serum albumin, transferrin, a carboxy-terminal peptide of chorionic gonadotropin (CG) β chain, a non-exact repeat peptide sequence, a polypeptide sequence composed of proline-alanine-serine polymer, an elastin-like peptide (ELP) repeat sequence), a homopolymer of glycine residues or a gelatin-like protein.

26. The method of any one of claims 19 to 23 wherein the polypeptide is in the form of a conjugate comprising a carbohydrate, a lipid, a liposome, a peptide, or an aptamer conjugated to the amino acid sequence comprising the epitope of dysfunctional P2X₇ receptor.

27. The method of any one of claims 19 to 26, wherein the moiety for enabling detection of the polypeptide is any suitable detectable moiety such a fluorescent moiety, a magnetic particle, a chromophore moiety, a phosphorescent moiety, a luminescent moiety, a light absorbing moiety, a radioactive moiety, and chemically detectable moieties like haptens, e.g. biotin, avidin, streptavidin and derivates thereof.

28. The method of claim 27, wherein when the polypeptide is labelled with a biotin moiety, the method comprises the step of contacting the cells (after step ii), with an anti-biotin antigen binding protein, preferably wherein the anti-biotin antigen binding protein comprises one or more moieties for enabling detection of the complex.

29. The method of claim 28, wherein the anti-biotin antigen binding protein is comprises a fluorophore.

30. The method of any one of claims 19 to 29, wherein the biological sample from a patient is a sample of peripheral blood, or a derivative thereof, such as serum, plasma or peripheral mononuclear monocyte (buffy coat) preparation.

31. A kit for use in a method according to any one of claims 19 to 30, the kit comprising:
a fusion protein or polypeptide capable of being bound by a receptor (eg a CAR) for binding to dysfunctional P2X₇ receptor;
optionally, one or more reagents for enabling detection of the fusion protein or polypeptide and complexes thereof.

32. The fusion protein of any one of claims 1 to 15, wherein the protein comprises the amino acid sequence as set forth in any of SEQ ID NOs: 145 to 158, 160 or 161.

33. The method of any one of claims 19 to 29, wherein the fusion protein comprises the amino acid sequence as set forth in any of SEQ ID NOs: 145 to 158, 160 or 161.


SEQUENCE LISTING (162 sequences)

SEQ ID NO: 1 - protein, 595 aa, synthetic construct
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 2 - protein, 17 aa, synthetic construct
GHNYTTRNILPGLNITC

SEQ ID NO: 3 - protein, 16 aa, synthetic construct
GHNYTTRNILPGLNIT

SEQ ID NO: 4 - protein, 15 aa, synthetic construct
KYYKENNVEKRTLIK

SEQ ID NO: 5 - protein, 17 aa, synthetic construct
KYYKENNVEKRTLIKVF

SEQ ID NO: 6 - protein, 25 aa, synthetic construct
GHNYTTRNILPGAGAKYYKENNVEK

SEQ ID NO: 7 - protein, 17 aa, synthetic construct
GHNYTTRNILPGLNITS

SEQ ID NO: 8 - protein, 21 aa, synthetic construct
GHNYTTRNILPGLNITSTFHK

SEQ ID NO: 9 - protein, 22 aa, synthetic construct
GHNYTTRNILPGLNITSTFHKT

SEQ ID NO: 10 - protein, 23 aa, synthetic construct
GHNYTTRNILPGLNITSTFHKTC

SEQ ID NO: 11 - protein, 27 aa, synthetic construct
GHNYTTRNILPGLNITSTFHKTSGSGK

SEQ ID NO: 12 - protein, 23 aa, synthetic construct
GHNYTTRNILPGLNITSTFHKTS

SEQ ID NO: 13 - protein, 16 aa, synthetic construct
DFPGHNYTTRNILPGC

SEQ ID NO: 14 - protein, 11 aa, synthetic construct
NYTTRNILPGL

SEQ ID NO: 15 - protein, 22 aa, synthetic construct
GHNYTTRNILPGLNITSGGGGS

SEQ ID NO: 16 - protein, 27 aa, synthetic construct
GHNYTTRNILPGLNITSGGGGSGGGGS

SEQ ID NO: 17 - protein, 32 aa, synthetic construct
GHNYTTRNILPGLNITSGGGGSGGGGSGGGGS

SEQ ID NO: 18 - protein, 24 aa, synthetic construct
GHNYTTRNILPGLNITSTFHKTGS

SEQ ID NO: 19 - protein, 22 aa, synthetic construct
GHNYTTRNILPGLNITSTFHGS

SEQ ID NO: 20 - protein, 19 aa, synthetic construct
GHNYTTRNILPGLNITSGS

SEQ ID NO: 21 - protein, 22 aa, synthetic construct
DFPGHNYTTRNILPGLNITSGS

SEQ ID NO: 22 - protein, 25 aa, synthetic construct
DFPGHNYTTRNILPGLNITSGGGGS

SEQ ID NO: 23 - protein, 30 aa, synthetic construct
DFPGHNYTTRNILPGLNITSGGGGSGGGGS

SEQ ID NO: 24 - protein, 35 aa, synthetic construct
DFPGHNYTTRNILPGLNITSGGGGSGGGGSGGGGS

SEQ ID NO: 25 - protein, 30 aa, synthetic construct
DFPGHNYTTRNILPGLNITSTFHKTSGSGK

SEQ ID NO: 26 - protein, 32 aa, synthetic construct
DFPGHNYTTRNILPGLNITSTFHKTSGSGKGS

SEQ ID NO: 27 - protein, 35 aa, synthetic construct
DFPGHNYTTRNILPGLNITSTFHKTSGSGKGGGGS

SEQ ID NO: 28 - protein, 28 aa, synthetic construct
DFPGHNYTTRNILPGLNITSTFHGGGGS

SEQ ID NO: 29 - protein, 25 aa, synthetic construct
GHNYTTRNILPGLNITSTFHGGGGS

SEQ ID NO: 30 - protein, 30 aa, synthetic construct
DFPGHNYTTRNILPGLNITSTFHKTGGGGS

SEQ ID NO: 31 - protein, 27 aa, synthetic construct
GHNYTTRNILPGLNITSTFHKTGGGGS

SEQ ID NO: 32 - protein, 27 aa, synthetic construct
GHNYTTRNILPGLNITSEPKSSDKTHT

SEQ ID NO: 33 - protein, 29 aa, synthetic construct
GHNYTTRNILPGLNITSGSEPKSSDKTHT

SEQ ID NO: 34 - protein, 32 aa, synthetic construct
GHNYTTRNILPGLNITSGGGGSEPKSSDKTHT

SEQ ID NO: 35 - protein, 37 aa, synthetic construct
GHNYTTRNILPGLNITSTFHKTSGSGKEPKSSDKTHT

SEQ ID NO: 36 - protein, 39 aa, synthetic construct
GHNYTTRNILPGLNITSTFHKTSGSGKGSEPKSSDKTHT

SEQ ID NO: 37 - protein, 42 aa, synthetic construct
GHNYTTRNILPGLNITSTFHKTSGSGKGGGGSEPKSSDKTHT

SEQ ID NO: 38 - protein, 29 aa, synthetic construct
GHNYTTRNILPGLNITSEPKSSDKTHTGS

SEQ ID NO: 39 - protein, 31 aa, synthetic construct
GHNYTTRNILPGLNITSGSEPKSSDKTHTGS

SEQ ID NO: 40 - protein, 34 aa, synthetic construct
GHNYTTRNILPGLNITSGGGGSEPKSSDKTHTGS

SEQ ID NO: 41 - protein, 39 aa, synthetic construct
GHNYTTRNILPGLNITSTFHKTSGSGKEPKSSDKTHTGS

SEQ ID NO: 42 - protein, 41 aa, synthetic construct
GHNYTTRNILPGLNITSTFHKTSGSGKGSEPKSSDKTHTGS

SEQ ID NO: 43 - protein, 44 aa, synthetic construct
GHNYTTRNILPGLNITSTFHKTSGSGKGGGGSEPKSSDKTHTGS

SEQ ID NO: 44 - protein, 32 aa, synthetic construct
GHNYTTRNILPGLNITSEPKSSDKTHTGGGGS

SEQ ID NO: 45 - protein, 34 aa, synthetic construct
GHNYTTRNILPGLNITSGSEPKSSDKTHTGGGGS

SEQ ID NO: 46 - protein, 37 aa, synthetic construct
GHNYTTRNILPGLNITSGGGGSEPKSSDKTHTGGGGS

SEQ ID NO: 47 - protein, 42 aa, synthetic construct
GHNYTTRNILPGLNITSTFHKTSGSGKEPKSSDKTHTGGGGS

SEQ ID NO: 48 - protein, 44 aa, synthetic construct
GHNYTTRNILPGLNITSTFHKTSGSGKGSEPKSSDKTHTGGGGS

SEQ ID NO: 49 - protein, 47 aa, synthetic construct
GHNYTTRNILPGLNITSTFHKTSGSGKGGGGSEPKSSDKTHTGGGGS

SEQ ID NO: 50 - protein, 30 aa, synthetic construct
DFPGHNYTTRNILPGLNITSEPKSSDKTHT

SEQ ID NO: 51 - protein, 32 aa, synthetic construct
DFPGHNYTTRNILPGLNITSGSEPKSSDKTHT

SEQ ID NO: 52 - protein, 35 aa, synthetic construct
DFPGHNYTTRNILPGLNITSGGGGSEPKSSDKTHT

SEQ ID NO: 53 - protein, 40 aa, synthetic construct
DFPGHNYTTRNILPGLNITSTFHKTSGSGKEPKSSDKTHT

SEQ ID NO: 54 - protein, 42 aa, synthetic construct
DFPGHNYTTRNILPGLNITSTFHKTSGSGKGSEPKSSDKTHT

SEQ ID NO: 55 - protein, 45 aa, synthetic construct
DFPGHNYTTRNILPGLNITSTFHKTSGSGKGGGGSEPKSSDKTHT

SEQ ID NO: 56 - protein, 32 aa, synthetic construct
DFPGHNYTTRNILPGLNITSEPKSSDKTHTGS

SEQ ID NO: 57 - protein, 34 aa, synthetic construct
DFPGHNYTTRNILPGLNITSGSEPKSSDKTHTGS

SEQ ID NO: 58 - protein, 37 aa, synthetic construct
DFPGHNYTTRNILPGLNITSGGGGSEPKSSDKTHTGS

SEQ ID NO: 59 - protein, 42 aa, synthetic construct
DFPGHNYTTRNILPGLNITSTFHKTSGSGKEPKSSDKTHTGS

SEQ ID NO: 60 - protein, 44 aa, synthetic construct
DFPGHNYTTRNILPGLNITSTFHKTSGSGKGSEPKSSDKTHTGS

SEQ ID NO: 61 - protein, 47 aa, synthetic construct
DFPGHNYTTRNILPGLNITSTFHKTSGSGKGGGGSEPKSSDKTHTGS

SEQ ID NO: 62 - protein, 35 aa, synthetic construct
DFPGHNYTTRNILPGLNITSEPKSSDKTHTGGGGS

SEQ ID NO: 63 - protein, 37 aa, synthetic construct
DFPGHNYTTRNILPGLNITSGSEPKSSDKTHTGGGGS

SEQ ID NO: 64 - protein, 40 aa, synthetic construct
DFPGHNYTTRNILPGLNITSGGGGSEPKSSDKTHTGGGGS

SEQ ID NO: 65 - protein, 45 aa, synthetic construct
DFPGHNYTTRNILPGLNITSTFHKTSGSGKEPKSSDKTHTGGGGS

SEQ ID NO: 66 - protein, 47 aa, synthetic construct
DFPGHNYTTRNILPGLNITSTFHKTSGSGKGSEPKSSDKTHTGGGGS

SEQ ID NO: 67 - protein, 50 aa, synthetic construct
DFPGHNYTTRNILPGLNITSTFHKTSGSGKGGGGSEPKSSDKTHTGGGGS

SEQ ID NO: 68 - protein, 40 aa, synthetic construct
DKTHTSPPSPAPELLGGGGSDFPGHNYTTRNILPGLNITS

SEQ ID NO: 69 - protein, 50 aa, synthetic construct
GGGGSEPKSSDKTHTSPPSPAPELLGGGGSDFPGHNYTTRNILPGLNITS

SEQ ID NO: 70 - protein, 5 aa, synthetic construct
GGGGS

SEQ ID NO: 71 - protein, 10 aa, synthetic construct
EPKSSDKTHT

SEQ ID NO: 72 - protein, 22 aa, synthetic construct
GSEPKSSDKTHTSPPSPAPELL

SEQ ID NO: 73 - protein, 30 aa, synthetic construct
GGGGSEPKSSDKTHTSPPSPAPELLGGGGS

SEQ ID NO: 74 - protein, 12 aa, synthetic construct
EPKSSDKTHTGS

SEQ ID NO: 75 - protein, 15 aa, synthetic construct
EPKSSDKTHTGGGGS

SEQ ID NO: 76 - protein, 15 aa, synthetic construct
EPKSSDKTHTSPPSP

SEQ ID NO: 77 - protein, 13 aa, synthetic construct
EPKSSDKTHTSPP

SEQ ID NO: 78 - protein, 14 aa, synthetic construct
EPKSSDKTHTSPPP

SEQ ID NO: 79 - protein, 10 aa, synthetic construct
EPKSSDKTHT

SEQ ID NO: 80 - protein, 10 aa, synthetic construct
DKTHTSPPSP

SEQ ID NO: 81 - protein, 8 aa, synthetic construct
DKTHTSPP

SEQ ID NO: 82 - protein, 12 aa, synthetic construct
EPKSDKTHTPPP

SEQ ID NO: 83 - protein, 14 aa, synthetic construct
EPKSDKTHTSPPSP

SEQ ID NO: 84 - protein, 15 aa, synthetic construct
EPKSGDKTHTGPPGP

SEQ ID NO: 85 - protein, 13 aa, synthetic construct
EPKSGDKTHTGPP

SEQ ID NO: 86 - protein, 14 aa, synthetic construct
EPKSGDKTHTGPPP

SEQ ID NO: 87 - protein, 10 aa, synthetic construct
EPKSGDKTHT

SEQ ID NO: 88 - protein, 10 aa, synthetic construct
DKTHTGPPGP

SEQ ID NO: 89 - protein, 8 aa, synthetic construct
DKTHTGPP

SEQ ID NO: 90 - protein, 14 aa, synthetic construct
EPKSDKTHTGPPGP

SEQ ID NO: 91 - protein, 15 aa, synthetic construct
EPKSGDKTHTSPPSP

SEQ ID NO: 92 - protein, 15 aa, synthetic construct
EPKSGDKTHTGPPSP

SEQ ID NO: 93 - protein, 15 aa, synthetic construct
EPKSSDKTHTGPPGP

SEQ ID NO: 94 - protein, 15 aa, synthetic construct
EPKSSDKTHTGPPSP

SEQ ID NO: 95 - protein, 13 aa, synthetic construct
EPKSSDKTHTGPP

SEQ ID NO: 96 - protein, 13 aa, synthetic construct
EPKSGDKTHTSPP

SEQ ID NO: 97 - protein, 15 aa, synthetic construct
EPKSTDKTHTTPPTP

SEQ ID NO: 98 - protein, 13 aa, synthetic construct
EPKSTDKTHTTPP

SEQ ID NO: 99 - protein, 14 aa, synthetic construct
EPKSTDKTHTTPPP

SEQ ID NO: 100 - protein, 10 aa, synthetic construct
EPKSTDKTHT

SEQ ID NO: 101 - protein, 10 aa, synthetic construct
DKTHTTPPTP

SEQ ID NO: 102 - protein, 8 aa, synthetic construct
DKTHTTPP

SEQ ID NO: 103 - protein, 14 aa, synthetic construct
EPKSDKTHTTPPTP

SEQ ID NO: 104 - protein, 15 aa, synthetic construct
EPKSSDKTHTTPPTP

SEQ ID NO: 105 - protein, 15 aa, synthetic construct
EPKSSDKTHTSPPTP

SEQ ID NO: 106 - protein, 15 aa, synthetic construct
EPKSADKTHTLPPMP

SEQ ID NO: 107 - protein, 15 aa, synthetic construct
EPKSVDKTHTLPPIP

SEQ ID NO: 108 - protein, 15 aa, synthetic construct
EPKSLDKTHTAPPAP

SEQ ID NO: 109 - protein, 13 aa, synthetic construct
EPKSVDKTHTAPP

SEQ ID NO: 110 - protein, 13 aa, synthetic construct
EPKSIDKTHTLPP

SEQ ID NO: 111 - protein, 10 aa, synthetic construct
DKTHTAPPLP

SEQ ID NO: 112 - protein, 10 aa, synthetic construct
DKTHTVPPLP

SEQ ID NO: 113 - protein, 12 aa, synthetic construct
EPKSSDKTHTSP

SEQ ID NO: 114 - protein, 15 aa, synthetic construct
EPKSCDKTHTCPPCP

SEQ ID NO: 115 - protein, 12 aa, synthetic construct
ERKCCVECPPCP

SEQ ID NO: 116 - protein, 12 aa, synthetic construct
ERKXXVEXPPXP

SEQ ID NO: 117 - protein, 10 aa, synthetic construct
ERKXXVEXPP

SEQ ID NO: 118 - protein, 7 aa, synthetic construct
VEXPPXP

SEQ ID NO: 119 - protein, 17 aa, synthetic construct
ELKTPLGDTTHTCPRCP

SEQ ID NO: 120 - protein, 17 aa, synthetic construct
ELKTPLGDTTHTXPRXP

SEQ ID NO: 121 - protein, 15 aa, synthetic construct
ELKTPLGDTTHTXPR

SEQ ID NO: 122 - protein, 25 aa, synthetic construct
DFPGHNYTTRNILPGLNITSTFHKT

SEQ ID NO: 123 - protein, 12 aa, synthetic construct
ELKTPLGDTTHT

SEQ ID NO: 124 - protein, 15 aa, synthetic construct
EPKSCDTPPPCPRCP

SEQ ID NO: 125 - protein, 15 aa, synthetic construct
EPKSXDTPPPXPRXP

SEQ ID NO: 126 - protein, 13 aa, synthetic construct
EPKSXDTPPPXPR

SEQ ID NO: 127 - protein, 10 aa, synthetic construct
EPKSXDTPPP

SEQ ID NO: 128 - protein, 10 aa, synthetic construct
DTPPPXPRXP

SEQ ID NO: 129 - protein, 12 aa, synthetic construct
ESKYGPPCPSCP

SEQ ID NO: 130 - protein, 12 aa, synthetic construct
EXKYGPPCPXCP

SEQ ID NO: 131 - protein, 9 aa, synthetic construct
EXKYGPPCP

SEQ ID NO: 132 - protein, 10 aa, synthetic construct
KYGPPCPXCP

SEQ ID NO: 133 - protein, 12 aa, synthetic construct
EPKSCDKTHTCP

SEQ ID NO: 134 - protein, 12 aa, synthetic construct
EPKSCDTPPPCP

SEQ ID NO: 135 - protein, 12 aa, synthetic construct
ESKYGPPSPSSP

SEQ ID NO: 136 - protein, 12 aa, synthetic construct
ERKSSVESPPSP

SEQ ID NO: 137 - protein, 12 aa, synthetic construct
EPKSSDTPPPSP

SEQ ID NO: 138 - protein, 5 aa, synthetic construct
TFHKT

SEQ ID NO: 139 - skipped

SEQ ID NO: 140 - protein, 20 aa, synthetic construct
DFPGHNYTTRNILPGLNITS

SEQ ID NO: 141 - protein, 17 aa, synthetic construct
EPKSCDKTHTSPPSPAP

SEQ ID NO: 142 - protein, 17 aa, synthetic construct
EPKSSDKTHTSPPSPAP

SEQ ID NO: 143 - protein, 5 aa, synthetic construct
GGGGS

SEQ ID NO: 144 - protein, 11 aa, synthetic construct
LEVLFQGPVRR

SEQ ID NO: 145 - protein, 266 aa, synthetic construct
DFPGHNYTTRNILPGLNITSTFHKTSGSGKGGGGSEPKSCDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 263 aa, synthetic construct
GHNYTTRNILPGLNITSTFHKTSGSGKGGGGSEPKSCDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 267 aa, synthetic construct
DFPGHNYTTRNILPGLNITSTFHKTSGSGKGGGGSEPKSSDKTHTSPPSPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 264 aa, synthetic construct
GHNYTTRNILPGLNITSTFHKTSGSGKGGGGSEPKSSDKTHTSPPSPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 266 aa, synthetic construct
DFPGHNYTTRNILPGLNITSTFHKTSGSGKGGGGSEPKSCDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 263 aa, synthetic construct
GHNYTTRNILPGLNITSTFHKTSGSGKGGGGSEPKSCDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 267 aa, synthetic construct
DFPGHNYTTRNILPGLNITSTFHKTSGSGKGGGGSEPKSSDKTHTCPPCPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 264 aa, synthetic construct
GHNYTTRNILPGLNITSTFHKTSGSGKGGGGSEPKSSDKTHTCPPCPAPEAARGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 277 aa, synthetic construct
DFPGHNYTTRNILPGLNITSTFHKTSGSGKGGGGSLEVLFQGPVRREPKSSDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 277 aa, synthetic construct
DFPGHNYTTRNILPGLNITSTFHKTSGSGKGGGGSLEVLFQGPVRREPKSSDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 267 aa, synthetic construct
DFPGHNYTTRNILPGLNITSTFHKTSGSGKGGGGSEPKSSDKTHTSPPSPAPEAARGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 267 aa, synthetic construct
DFPGHNYTTRNILPGLNITSTFHKTSGSGKGGGGSEPKSSDKTHTCPPCPAPEAARGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 266 aa, synthetic construct
DFPGHNYTTRNILPGLNITSTFHKTSGSGKGGGGSEPKSSDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 267 aa, synthetic construct
DFPGHNYTTRNILPGLNITSTFHKTSGSGKGGGGSEPKSSDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 159 - protein, 232 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 160 - protein, 267 aa, synthetic construct
DFPGHNYTTRNILPGLNITSTFHKTSGSGKGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 161 - protein, 267 aa, synthetic construct; identical to SEQ ID NO: 160

SEQ ID NO: 162 - protein, 232 aa, synthetic construct
EPKSSDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKELTKNQVSLTCLVKGFYPSDIAVEWESNGQPENNYKTTPPVLKSDGSFFLYSKLTVDKSRWQQGNVFSCSVMHEALHNHYTQKSLSLSPGK